(19) United States
(12) Patent Application Publication
(10) Pub. No.: US 2024/0382189 A1
(43) Pub. Date: Nov. 21, 2024

(54) FIXED SWAGED SOFT SUTURE METHODS & APPARATUS

(71) Applicant: RIVERPOINT MEDICAL, LLC, Portland, OR (US)

(72) Inventors: Elliot BIXBY, Milwaukie, OR (US); Brenna Popham, Bend, OR (US)

(21) Appl. No.: 18/666,753

(22) Filed: May 16, 2024

Related U.S. Application Data
(60) Provisional application No. 63/466,961, filed on May 16, 2023.
(60) Provisional application No. 63/529,191, filed on Jul. 27, 2023.
(60) Provisional application No. 63/585,090, filed on Sep. 25, 2023.

(51) Int. Cl. A61B 17/04

(52) U.S. Cl. CPC A61B 17/0401; A61B 2017/0445

(57) ABSTRACT

Apparatus, methods and systems for attaching soft tissue to bone are disclosed. A suture anchor assembly includes an anchor and a first strand. The first strand may be locked relative to the anchor to enable the tensioning of the strands to compress the anchor and set it within a bone socket, approximate the tissue and compress the tissue against the bone. The suture anchor assembly may further comprise a second flexible strand, the second flexible strand may be slidable relative to the anchor.

### CROSS-REFERENCE TO RELATED APPLICATIONS

This application claims the benefit of U.S. Provisional Appl. No. 63/466,961, filed on May 16, 2023, entitled “Fixating a Suture Strand Method & Apparatus,” U.S. Provisional Appl. No. 63/529, 191, filed on Jul. 27, 2023, entitled “Fixed Swaged Soft Suture Methods & Apparatus,” and U.S. Provisional Appl. No. 63/585,090, filed on Sep. 25, 2023, entitled “All Suture Anchor Fixed Strand Construct & Methods,” which are all incorporated by reference in their entireties.

### TECHNICAL FIELD

The disclosure relates to methods, devices, and systems for a surgical device used to attach tissue to bone. More particularly, the disclosure relates to a suture anchoring system that can be used to attach tissue to bone for various orthopedic repair applications.

### BACKGROUND OF THE INVENTION

Soft tissue may tear away from bone during vigorous exercise or sporting activities and various types of orthopedic procedures are often performed to repair musculoskeletal injuries. For example, rotator cuff tears are one common condition causing shoulder disability in patients. Failure of the rotator cuff tendon (e.g., detachment from the bone or soft tissue tears) may result in pain in the shoulder and/or loss of shoulder function. When such injuries occur, reattachment is often necessary to repair the damaged tissue. Several different arthroscopic repair techniques are known for repairing rotor cuff tears, including single-row, double-row and bridging techniques. Suture anchoring systems are one type of surgical device that has been developed to facilitate these repairs. Further improvements or advancements in suture anchoring systems are required.

### BRIEF SUMMARY OF THE INVENTION

In one embodiment, the suture anchoring systems comprise a fixed or first strand and an anchor. The suture anchoring system may further comprise a sliding strand or second strand. The suture anchoring system may further comprise a third strand. The anchor can be slidably engaged with a portion of the second strand, enabling the second strand to be movable relative to the anchor. The anchor is also engaged with a portion of the first strand, enabling the first strand to be locked or substantially locked relative to the anchor. Substantially locked allows the first strand to move or slide a slight amount before it locks or becomes non-movable relative to the anchor. Substantially locked may comprise movement from a first position, the first position being an unlocked position, to a second position, the second position being a locked position. The anchor may further be engaged with a third strand. The third strand may be sliding or movable relative to the anchor or the third strand may help fixate the first strand to enable “locking” or “substantially locked” positioning.

In one embodiment, at least one method of making the suture anchor system comprises the steps of: weaving a portion of a second strand through at least a portion of an anchor in a first weave pattern to create one or more first external segments, the first one or more first external segments extend from or contact the anchor outer surface; weaving a portion of a third strand through at least a portion of an anchor and ta first strand in a second weave pattern to create one or more second external segments, the one or more second external segments extend from or contact a portion of the first strand surface; encircling a portion of the first strand around the outer surface of the anchor; and intersecting the first free end and the second free end of the third strand to create one or more intersecting points, the intersecting points comprises locking intersecting points.

### BRIEF DESCRIPTION OF THE SEVERAL VIEWS OF THE DRAWINGS

FIGS. 1A-1C depict various representative plan views of a first embodiment of a suture anchor system and a method of making the suture anchor system;

FIG. 1D depicts an isometric side view of the alternative dual piercing of the suture strand with the anchor for the first embodiment of FIGS. 1A-1C;

FIGS. 2A-2D depict various plan views of the suture anchor system and the method of making the suture anchor system of FIGS. 1A-1C;

FIGS. 3A-3C depict various plan views of a second embodiment of a suture anchor system and a method of making the second embodiment suture anchor system;

FIGS. 4A-4C depict various plan views of a third embodiment of a suture anchor system and method of making the third embodiment suture anchor system;

FIGS. 5A-5D depict various plan views of a fourth embodiment of a suture anchor system and a method of making the fourth embodiment suture anchor system;

FIGS. 6A-6B depict front views of a fifth embodiment of a suture anchor system with an inserter;

FIGS. 7A-7D illustrates the fifth embodiment suture anchor system and method to create the fifth embodiment suture anchor assembly of FIGS. 6A-6B;

FIGS. 8A-8M illustrates the fifth embodiment suture anchor system and an alternate method to create the fifth embodiment suture anchor assembly of FIGS. 6A-6B;

FIGS. 9A-9E illustrates ultimate load testing results data for a control and different configurations of the fifth embodiment suture anchor system of FIGS. 6A-6B;

FIGS. 10A-10H depicts a sixth embodiment suture anchor system and a method of making a sixth embodiment of a suture anchor system;

FIGS. 11A-11E depicts a seventh suture anchor system and method of making the seventh embodiment of a suture anchor system;

FIGS. 12A-12G depicts an eighth embodiment of a suture anchor system and a method of making an eighth embodiment of a suture anchor system;

FIGS. 13A-13C depicts a ninth embodiment of a suture anchor system and a method of making a ninth embodiment of a suture anchor system;

FIGS. 14-15 illustrates a flowchart with a method to create the suture anchor system of FIG. 1A-1D and 2A-2D;

FIG. 16 illustrates a flowchart with a method to create the suture anchor system of FIGS. 3A-3C;

FIG. 17 illustrates a flowchart with method to create the suture anchor system of FIGS. 4A-4C;

FIG. 18 illustrates a flowchart with a method to create the suture anchor system of FIGS. 5A-5D;

FIGS. 19A-19B illustrates a flowchart of the method to create the suture anchor system of FIGS. 6A-6B;

FIG. 20 illustrates a flowchart of the method to create the suture anchor system of FIGS. 10A-10H;

FIG. 21 illustrates a flowchart of the method to create the suture anchor system of FIGS. 11A-11E;

FIG. 22 illustrates a flowchart of the method to create the suture anchor system of FIGS. 12A-12G;

FIG. 23 illustrates a flowchart of the method to create the suture anchor system of FIGS. 13A-13C; and

FIGS. 24A-24E illustrate one embodiment of a method of surgical use.

### DETAILED DESCRIPTION OF THE INVENTION

The disclosure relates to devices, methods and systems of different embodiments of suture anchoring systems 5, 105, 110, 125, 128, 150, 210, 215, 220, 225 used in orthopedic procedures to attach soft tissue to bone. The suture anchoring systems 5, 105, 110, 125, 128, 150, 210, 215, 220, 225 comprise “soft or flexible” materials to facilitate a portion of the assembly to slide, lock, compress, expand or change shape within a socket or hole for proper fixation.

The suture anchoring systems are configured for a variety of orthopedic procedures or applications, including rotator cuff repairs, achilles tendon repairs, patellar tendon repairs, ACL//PCL reconstructions, hip reconstructions, shoulder reconstructions, and/or any combination thereof. Furthermore, different arthroscopic techniques can be used, including single-row, double-row, and bridging techniques.

In one embodiment, the suture anchoring systems 5, 105, 110, 125, 128, 150, 210, 215, 220, 225 comprise a fixed or first strand 10 and an anchor 30. The suture anchoring system may further comprise a sliding strand or second strand 80. The anchor 30 is slidably engaged with a portion of the second strand 80, and the second strand 80 is movable relative to the anchor 30. The anchor 30 is also engaged with a portion of the first strand 10, and locked or substantially locked relative to the fixed strand or first strand 10. Substantially locked allows the first strand to move or slide a slight amount before it locks or becomes non-movable relative to the anchor. Substantially locked may comprise movement from a first position, the first position being an unlocked position, to a second position, the second position being a locked position. The suture anchoring system 5, 105, 110, 125, 128, 150, 210, 215, 220, 225 may further comprise a third strand. The third strand may be an extra strand or a fixation strand. The suture anchoring systems 5, 105, 110, 125, 128, 150, 210, 215, 220, 225 may further comprise an inserter or delivery device 155. The third strand may be locked or sliding relative to the anchor.

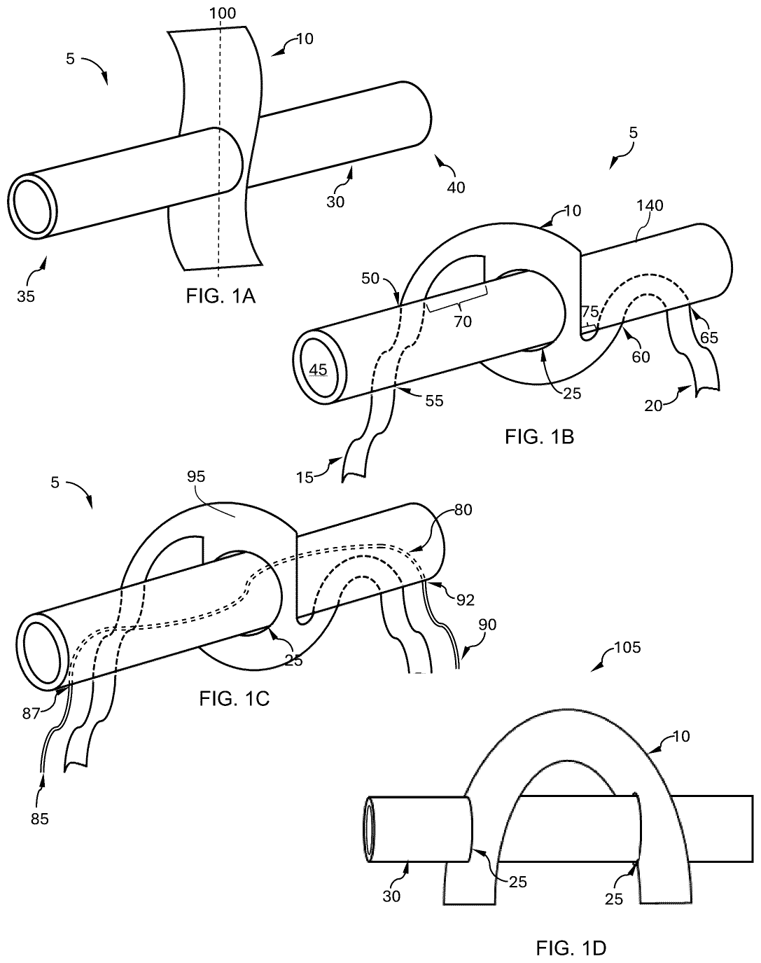

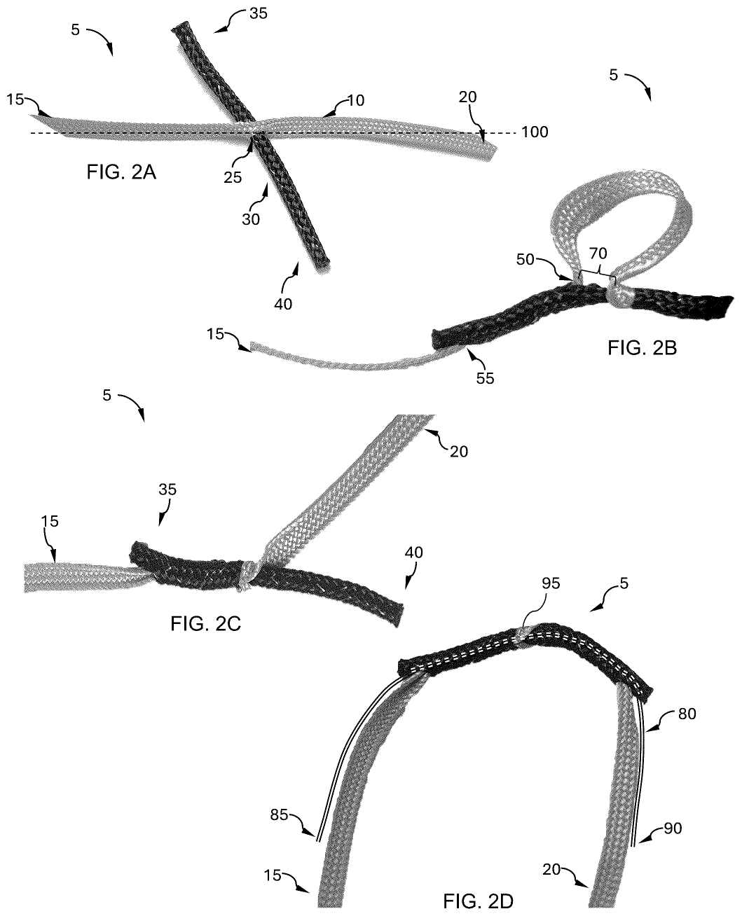

FIGS. 1A-1C and 2A-2D depict one embodiment of a suture anchoring system 5. The suture anchor system 5 comprises a first strand 10 and an anchor 30. The suture anchor system 5 may further comprise a second strand 80. The suture anchor system 5 may further comprise a third strand 120. The first strand 10 comprises a first strand longitudinal axis 100, a first free end 15 and a second free end 20, the first strand comprising a first strand opening or piercing 25, the first strand opening or piercing 25 positioned between the first free end 15 and the second free end 20. The second strand 80 comprises a first free end 85 and a second free end 90. The third strand 120 comprises a first free end 160 and a second free end 165.

The anchor 30 comprising a tubular anchor body, a first end 35, a second end 40, a lumen 45, and an anchor material. The anchor 30 may further comprise an outer surface 140. The anchor 30 may further comprise a first sidewall (not shown) and a second sidewall (not shown). The lumen 45 extending between the first end 35 and second end 40 of the anchor 30. The anchor 30 being engaged and/or slidably inserted into the first strand opening 25 of the first strand 10 at a set location or position on the anchor 30. The anchor 30 being inserted transverse to the first strand longitudinal axis 100 through the first strand opening 25.

At least a portion of the first free end 15 extends through the anchor 30 outer surface 140 at a first location 50 that is spaced apart 70 from the set location and/or the first strand opening 25, and exits out of the outer surface 140 of the anchor 30 at a second location 55. The second free end 20 extends through the outer surface 140 of the anchor 30 at a third location 60 that is spaced apart 75 from the set location and/or the first strand opening 25, and exits out the outer surface 140 of the anchor 30 at a fourth location 65. The first location and the second location are positioned adjacent or near the first end 35 of the anchor 30. The third location 60 and the fourth location 65 are positioned adjacent or near the second 40 of the anchor 30. The distance of the spacing apart 70, 75 may comprise equal to or greater than 1 mm; may comprise equal to or greater than 2 mm; may comprise equal to or greater than 3 mm; may comprise equal to or greater than 4 mm; may comprise equal to or greater than 5 mm; and/or may comprise a range of 3 mm to 8 mm. The lower the distance to the set location or first strand opening 25 creates a more significant “lock,” preventing the first strand 10 to move relative to the anchor 30. The higher the distance to the set location and/or first strand opening 25 allows some movement of the first strand 10 to move relative to the anchor 30 before it “locks” into position.

The first location 50 and the third location 60 are positioned 180 degrees apart or directly opposite each other. The first location 50 and the second location 55 are positioned 180 degrees apart or directly opposite each other. The third location 60 and the fourth location 65 align along the same axis and/or are on the same plane. Alternatively, the first location 50 and the third location are positioned or aligned along the same axis and/or on the same plane.

The second strand 80 may comprise a first free end 85 and a second free end 90. The second strand 80 extends or pierces through the outer surface 140 of the anchor 30 at a fifth location 87 into the lumen 45 and extends parallel to the lumen 45 to exit out of the anchor 30 at a sixth location 92. The fifth location 87 is positioned adjacent or near the first end 35 of the anchor 30 and the sixth location 92 is positioned adjacent or near the second end 40 of the anchor 30. The fifth location 92 is spaced apart from the second location 55 and the sixth location 92 is spaced apart from the fourth location. Alternatively, a portion of the second strand 80 extends parallel to the lumen 45 of the anchor with the first free end 85 exiting a sidewall of the anchor 30 at a first end 35 of the anchor 30 and the second free end 90 exiting a sidewall of the anchor 30 at a second 35 of the anchor 30.

FIG. 1D depicts an alternative suture anchoring system 105 that deviates slightly from FIGS. 1A-1C and 2A-2D. The suture anchor system 105 comprises a first strand 10 and an anchor 30. The suture anchor system 105 may further comprise a second strand 80. The suture anchor system 105 may further comprise a third strand 120. The suture anchor system 105 comprises: a first strand 10, the first strand 10 comprises a first strand longitudinal axis 100, a first free end 15 and a second free end 20, the first strand 10 comprising a plurality of first strand openings or piercings 25, the plurality of first strand openings or piercings 25 positioned between the first free end 15 and the second free end 20 of the first strand.

The anchor 30 comprising a tubular anchor body, a first end 35, a second end 40, a lumen 45, and an anchor material. The anchor 30 may further comprise an outer surface 140. The anchor 30 may further comprise a first sidewall (not shown) and a second sidewall (not shown). The lumen 45 extending between the first end 35 and second end 40 of the anchor 30. The anchor 30 being engaged and/or slidably inserted into each of the plurality of first strand openings 25 of the first strand 10 at a set location or position on the anchor 30. The anchor 30 being inserted transverse to the first strand longitudinal axis 100 through the plurality of first strand openings 25.

At least a portion of the first free end 15 extends through the anchor 30 outer surface 140 at a first location 50 that is spaced apart 70 from the set location and/or the plurality of first strand openings 25, and exits out of the outer surface 140 of the anchor 30 at a second location 55. The second free end 20 extends through the outer surface 140 of the anchor 30 at a third location 60 that is spaced apart 75 from the set location and/or the plurality of first strand openings 25, and exits out the outer surface 140 of the anchor 30 at a fourth location 65. The first location and the second location are positioned adjacent or near the first end 35 of the anchor 30. The third location 60 and the fourth location 65 are positioned adjacent or near the second 40 of the anchor 30. The distance of the spacing apart 70, 75 may comprise equal to or greater than 1 mm; may comprise equal to or greater than 2 mm; may comprise equal to or greater than 3 mm; may comprise equal to or greater than 4 mm; may comprise equal to or greater than 5 mm; and/or may comprise a range of 3 mm to 8 mm. The lower the distance to the set location or first strand opening 25 creates a more significant “lock,” preventing the first strand 10 to move relative to the anchor 30. The higher the distance to the set location and/or first strand opening 25 allows some movement of the first strand 10 to move relative to the anchor 30 before it “locks” into position.

The first location 50 and the third location 60 are positioned 180 degrees apart or directly opposite each other. The first location 50 and the second location 55 are positioned 180 degrees apart or directly opposite each other. The third location 60 and the fourth location 65 align along the same axis and/or are on the same plane. Alternatively, the first location 50 and the third location are positioned or aligned along the same axis and/or on the same plane.

The second strand 80 may comprise a first free end 85 and a second free end 90. The second strand 80 extends or pierces through the outer surface 140 of the anchor 30 at a fifth location 87 into the lumen 45 and extends parallel to the lumen 45 to exit out of the anchor 30 at a sixth location 92. The fifth location 87 is positioned adjacent or near the first end 35 of the anchor 30 and the sixth location 92 is positioned adjacent or near the second end 40 of the anchor 30. The fifth location 92 is spaced apart from the second location 55 and the sixth location 92 is spaced apart from the fourth location. Alternatively, a portion of the second strand 80 extends parallel to the lumen 45 of the anchor with the first free end 85 exiting a sidewall of the anchor 30 at a first end 35 of the anchor 30 and the second free end 90 exiting a sidewall of the anchor 30 at a second 35 of the anchor.

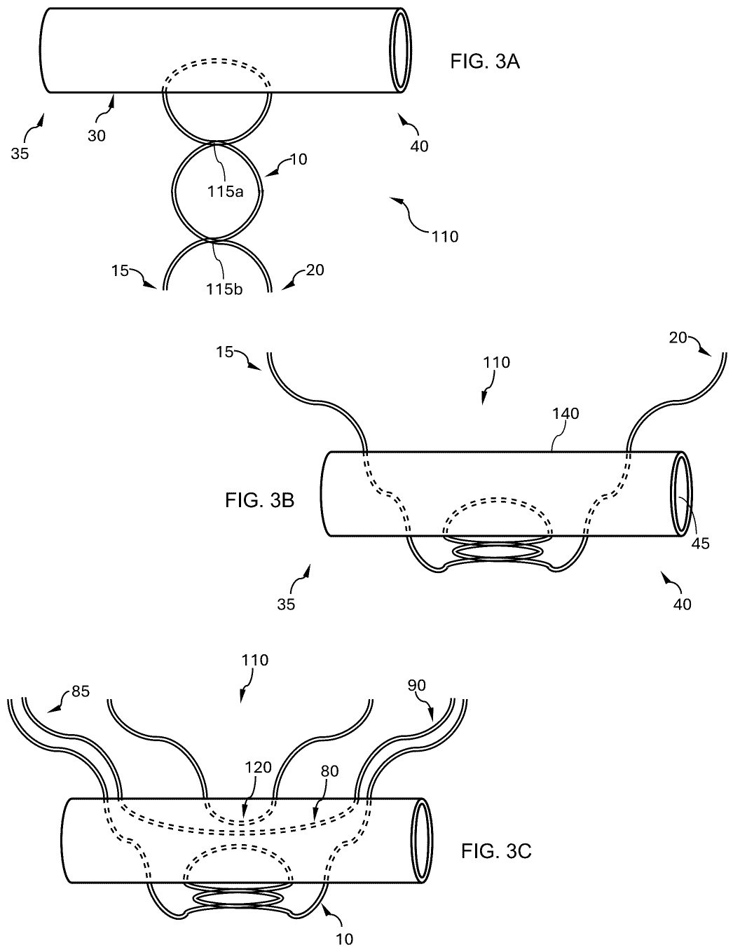

FIGS. 3A-3C depict various plan views of a second embodiment of a suture anchoring system 110 and a method of making the second embodiment suture anchoring system 110. The suture anchor system 110 comprises a first strand 10 and an anchor 30. The suture anchor system 110 may further comprise a second strand 80. The suture anchor system 110 may further comprise a third strand 120. The first strand 10 comprises a first strand longitudinal axis 100, a first free end 15 and a second free end 20. The first strand 10 further comprising a plurality of first strand intersections 115a, 115b. The anchor 30 comprising a tubular anchor body, a first end 35, a second end 40, a lumen 45, and an anchor material. The anchor 30 may further comprise an outer surface 140. The anchor 30 may further comprise a first sidewall (not shown) and a second sidewall (not shown). The lumen 45 extending between the first end 35 and second end 40 of the anchor 30.

At least a portion of the first strand 10 is positioned or disposed within the lumen 45 of the anchor 30. A first free end 15 of the first strand 10 extends from the outer surface 140 of the anchor 30 at a first location or position and the second free end 20 of the first strand 10 extends from the outer surface 140 of the anchor 30 at a second location or position. The first location is spaced apart from the second location. The first free end 15 and the second free end 20 of the first strand 10 intersect to create one or more intersecting points or regions 115a, 115b. The one or more intersecting points or regions 115a, 115b may comprise locking one or more intersecting points or regions 115a, 115b. The locking one or more intersecting points or regions may comprise a locking splice, the locking splice may comprise Brummel splices. The one or more intersecting points or regions 115a, 115b may be adjacent to or immediately adjacent to the outer surface 140 of the anchor 30. Alternatively, at least a portion of the one or more intersecting points or regions 115a, 115b may contact a portion of the outer surface 140 of the anchor 30.

In another embodiment, at least a portion of the first strand 10 is positioned or disposed within the lumen 45 of the anchor 30. A first free end 15 of the first strand 10 extends through the anchor 30 to exit at a first location or position. The second free end 20 of the first strand 10 extends through the anchor 30 to exit at a second location or position. A first free end 15 of the first strand 10 extends through the anchor 30 to exit at a first location or position may extend transverse to a horizontal axis of the anchor 30 and/or it may extend obliquely relative to the horizontal axis of the anchor 30. The second free end 20 of the first strand 10 extends through the anchor 30 to exit at a second location or position may extend transverse to the horizontal axis of the anchor 30 and/or it may extend obliquely relative to the horizontal axis of the anchor. The first location is spaced apart from the second location. The first position or location is at, near or adjacent to the first end 35 of the anchor 30. The second position or location is at, near or adjacent to the second end 40 of the anchor 30.

The first free end 15 and the second free end 20 of the first strand 10 intersect to create one or more intersecting points or regions 115a, 115b. The one or more intersecting points or regions 115a, 115b may comprise locking one or more intersecting points or regions 115a, 115b. The locking one or more intersecting points or regions may comprise a locking splice, the locking splice may comprise Brummel splices. The one or more intersecting points or regions 115a, 115b may be adjacent to or immediately adjacent to the outer surface 140 of the anchor 30. Alternatively, at least a portion of the one or more intersecting points or regions 115a, 115b may contact a portion of the outer surface 140 of the anchor 30.

At least a portion of the second strand 80 is disposed within the lumen 45 of the anchor 30. At least a portion of the second strand 80 extends through the lumen 45 of the anchor 30. At least a portion of the second strand 80 extends parallel through the lumen 45 of the anchor. At least a portion of the second strand 80 is disposed within the lumen 45 and aligns or follows the horizontal axis of the anchor 30. A first free end 85 of the second strand 80 extends from the outer surface 140 of the anchor 30 from a third position or location and the second free end 90 of the second strand extends from the outer surface 140 of the anchor 30 from a fourth position or location. The third position or location is spaced apart from the fourth position or location. The third position or location and the fourth position or location is disposed between the first position or location and the second position or location. The third position or location is at, near or adjacent to the first end 35 of the anchor 30. The fourth position or location is at, near or adjacent to the second end 40 of the anchor 30.

At least a portion of the third strand 120 is disposed within the lumen 45 of the anchor 30. At least a portion of the third strand 120 extends through the lumen 45 of the anchor 30. At least a portion of the third strand 120 extends parallel through the lumen 45 of the anchor. At least a portion of the third strand 120 is disposed within the lumen 45 and aligns or follows the horizontal axis of the anchor 30. A first free end 85 of the third strand 120 extends from the outer surface 140 of the anchor 30 from a fifth position or location and the second free end 90 of the third strand 120 extends from the outer surface 140 of the anchor 30 from a sixth position or location. The fifth position or location is spaced apart from the sixth position or location. The fifth position or location and the sixth position or location is disposed between the third position or location and the fourth position or location. The fifth position or location is at, near or adjacent to the first end 35 of the anchor 30. The sixth position or location is at, near or adjacent to the second end 40 of the anchor 30. The second strand 80 and/or the third strand 120 may be movable or slidable relative to the anchor 30.

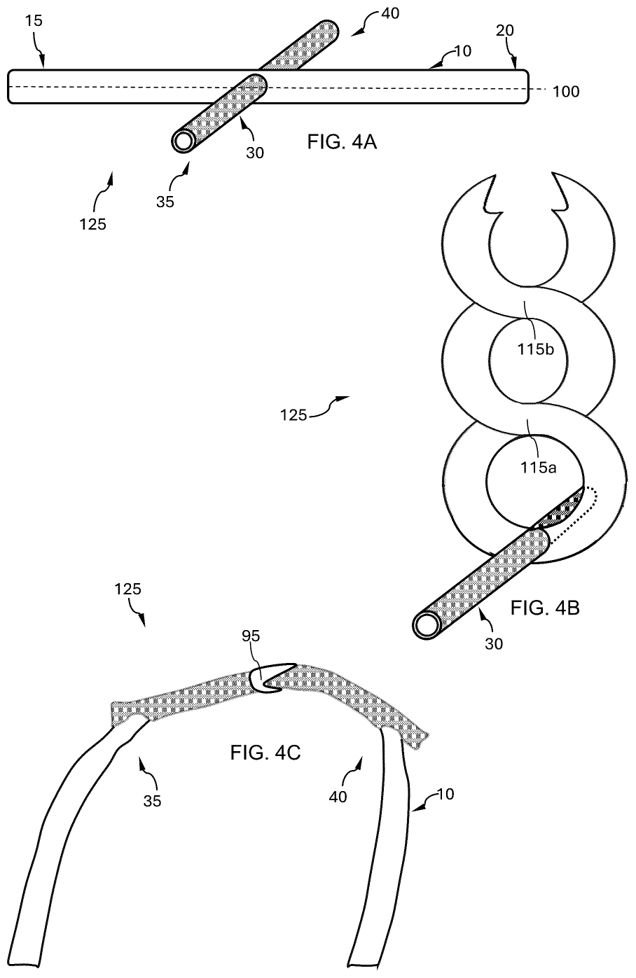

FIGS. 4A-4C depict various plan views of a third embodiment of a suture anchor system 125 and method of making the third embodiment suture anchor system 125. The suture anchor system 125 comprises a first strand 10 and an anchor 30. The suture anchor system 125 may further comprise a second strand 80. The suture anchor system 125 may further comprise a third strand 120. The first strand 10 comprises a first strand longitudinal axis 100, a first free end 15 and a second free end 20. The first strand 10 further comprising at least one first strand opening 25. The second strand 80 comprises a first free end 85 and a second free end 90. The third strand 120 comprises a first free end 160 and a second free end 165.

The anchor 30 comprising a tubular anchor body, a first end 35, a second end 40, a lumen 45, and an anchor material. The anchor 30 may further comprise an outer surface 140. The anchor 30 may further comprise a first sidewall (not shown) and a second sidewall (not shown). The lumen 45 extending between the first end 35 and second end 40 of the anchor 30. The anchor 30 being engaged and/or slidably inserted into the at least one first strand opening 25 of the first strand 10 at a set location or position on the anchor 30. The anchor 30 being inserted transverse to the first strand longitudinal axis 100 through the at least one first strand opening 25.

The first free end 15 and the second free end 20 of the first strand 10 intersect to create one or more intersecting points or regions 115a, 115b. The one or more intersecting points or regions 115a, 115b may comprise locking one or more intersecting points or regions 115a, 115b. The locking one or more intersecting points or regions may comprise a locking splice, the locking splice may comprise Brummel splices. The one or more intersecting points or regions 115a, 115b may be adjacent to or immediately adjacent to the outer surface 140 of the anchor 30. Alternatively, at least a portion of the one or more intersecting points or regions 115a, 115b may contact a portion of the outer surface 140 of the anchor 30. At least a portion of the one or more intersecting points or regions 115a, 115b may contact a portion of the outer surface 140 of the anchor 30 and/or encircle the outer surface 140 of the anchor 30 to create a locking collar 95. The first strand 10 may be locked or substantially locked relative to anchor 30.

A first free end 15 of the first strand 10 extends through the anchor 30 to exit at a first location or position. The second free end 20 of the first strand 10 extends through the anchor 30 to exit at a second location or position. A first free end 15 of the first strand 10 extends through the anchor 30 to exit at a first location or position may extend transverse to a horizontal axis of the anchor 30 and/or it may extend obliquely relative to the horizontal axis of the anchor 30. The second free end 20 of the first strand 10 extends through the anchor 30 to exit at a second location or position may extend transverse to the horizontal axis of the anchor 30 and/or it may extend obliquely relative to the horizontal axis of the anchor. The first location is spaced apart from the second location. The first position or location is at, near or adjacent to the first end 35 of the anchor 30. The second position or location is at, near or adjacent to the second end 40 of the anchor 30.

The suture anchor system 125 further comprises a second strand 80 (not shown). At least a portion of the second strand 80 is disposed within the lumen 45 of the anchor 30. At least a portion of the second strand 80 extends through the lumen 45 of the anchor 30. At least a portion of the second strand 80 extends parallel through the lumen 45 of the anchor. At least a portion of the second strand 80 is disposed within the lumen 45 and aligns or follows the horizontal axis of the anchor 30. A first free end 85 of the second strand 80 extends from the outer surface 140 of the anchor 30 from a third position or location and the second free end 90 of the second strand 80 extends from the outer surface 140 of the anchor 30 from a fourth position or location. The second strand 80 may be slidable or movable relative to the anchor 30.

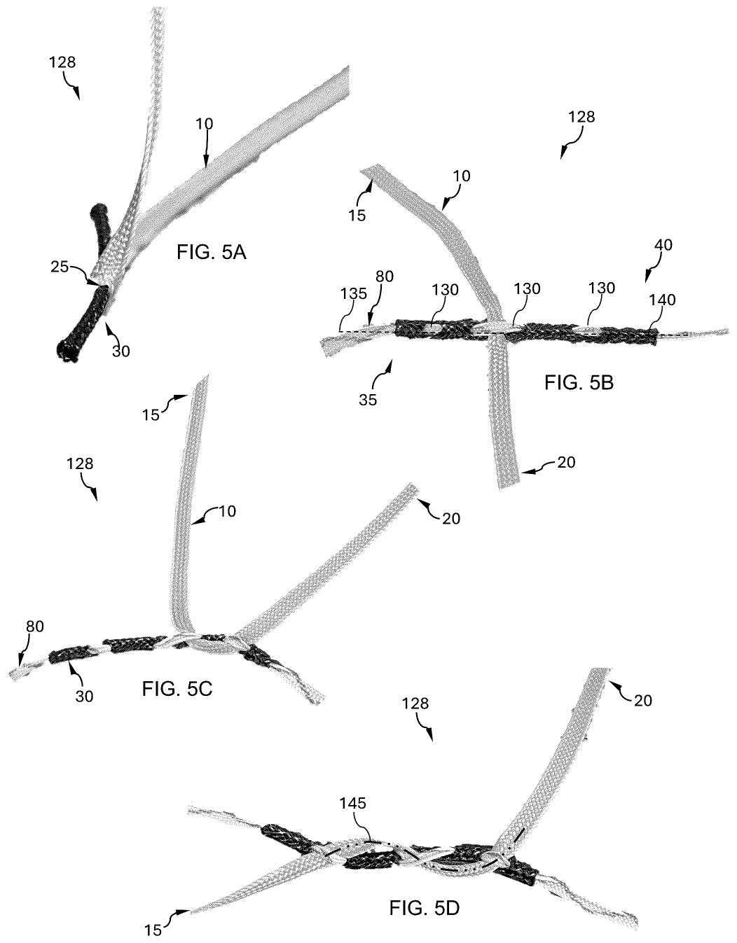

FIGS. 5A-5D depict various plan views of a fourth embodiment of a suture anchor system 128 and a method of making the fourth embodiment suture anchor system 128. The suture anchor system 128 comprises a first strand 10 and an anchor 30. The suture anchor system 128 may further comprise a second strand 80. The suture anchor system 125 may further comprise a third strand 120. The first strand 10 comprises a first strand longitudinal axis 100, a first free end 15 and a second free end 20. The first strand 10 further comprising at least one first strand opening 25.

The anchor 30 comprising a tubular anchor body, a first end 35, a second end 40, a lumen 45, and an anchor material. The anchor 30 may further comprise an outer surface 140. The anchor 30 may further comprise a first sidewall (not shown) and a second sidewall (not shown). The lumen 45 extending between the first end 35 and second end 40 of the anchor 30. The anchor 30 being engaged and/or slidably inserted into the at least one first strand opening 25 of the first strand 10 at a set location or position on the anchor 30. The anchor 30 being inserted transverse to the first strand longitudinal axis 100 through the at least one first strand opening 25.

At least a portion of the second strand 80 extending over the outer surface 140 and through a portion of the lumen 45 of the anchor 30 at one or more set locations to create one or more loop segments 130, the one or more loop segments 130 contact or are adjacent to the outer surface 140 of the anchor 30. The one or more loop segments 130 are aligned, substantially aligned, and/or parallel to the longitudinal axis of the anchor 30. In another embodiment, at least a portion of the second strand 80 extending into the lumen 45 and exiting out of a sidewall of the anchor 30 to create one or more loop segments 130. In one exemplary embodiment, the suture anchor system 128 comprises at least three loop segments 130.

At least a portion of the first strand 10 weaving through or intersecting at least one of the one or more loop segments 130. At least a portion of the first strand 10 extending through at least one the one or more loop segments 130. At least a portion of the first strand 10 weaving through or intersecting each the one or more loop segments 130. At least a portion of the first strand 10 extending through each of the one or more loop segment 130s. The weaving, intersecting and/or extending through may follow a weave pattern 145. The weave pattern 145 may comprise an “S” weave shaped pattern. The weaving pattern 145 allows at least a portion of the second strand 10 to contact a portion of the outer surface 140 of the anchor 30 to facilitate or allow the first strand 10 to be locked relative to the anchor 30 by cinching, wedging or frictionally engaging to the anchor 30.

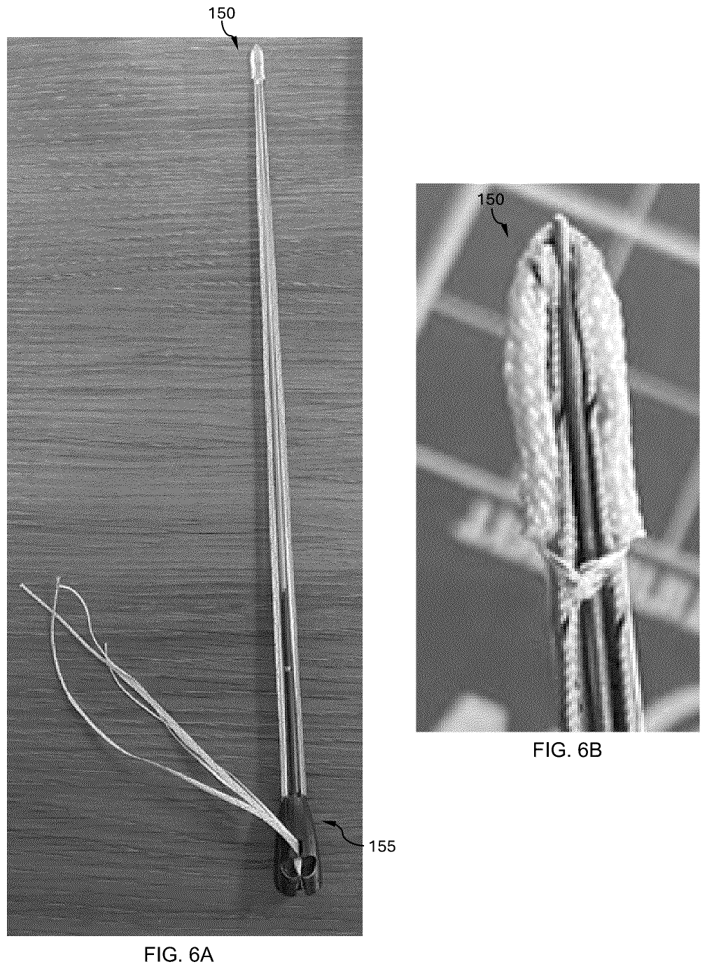

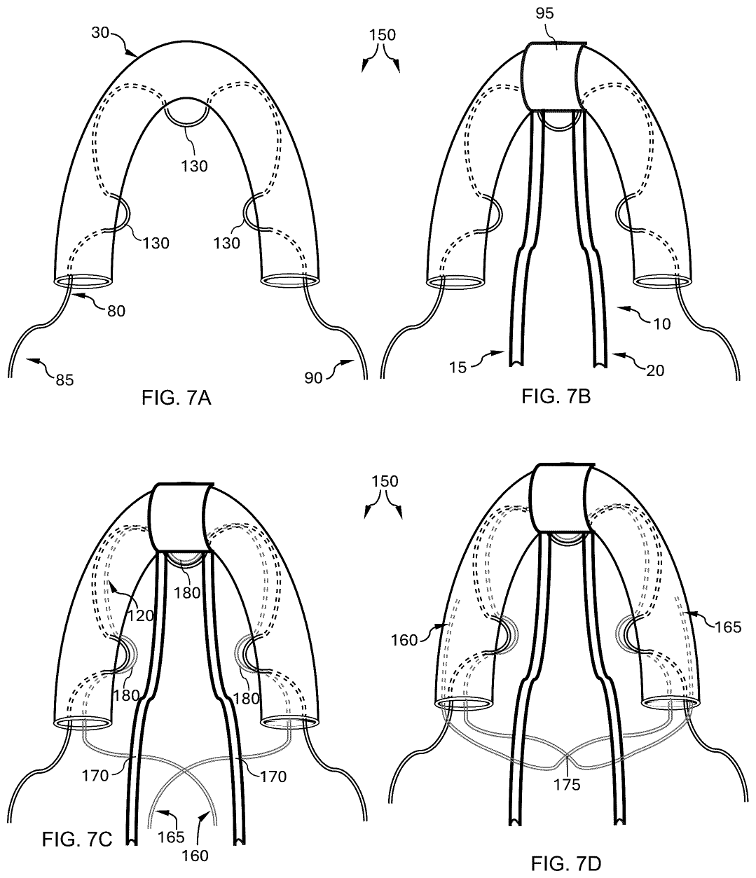

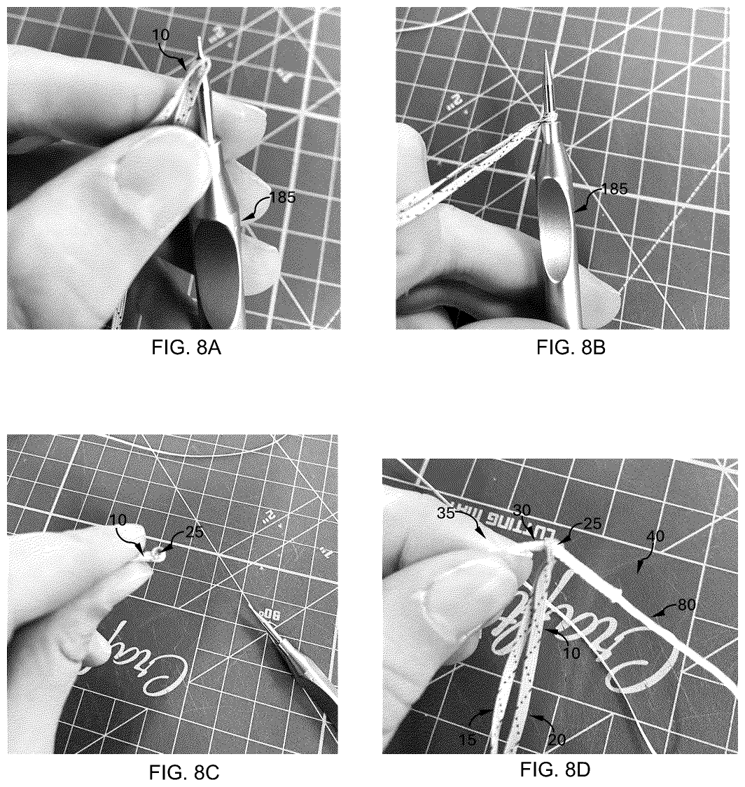

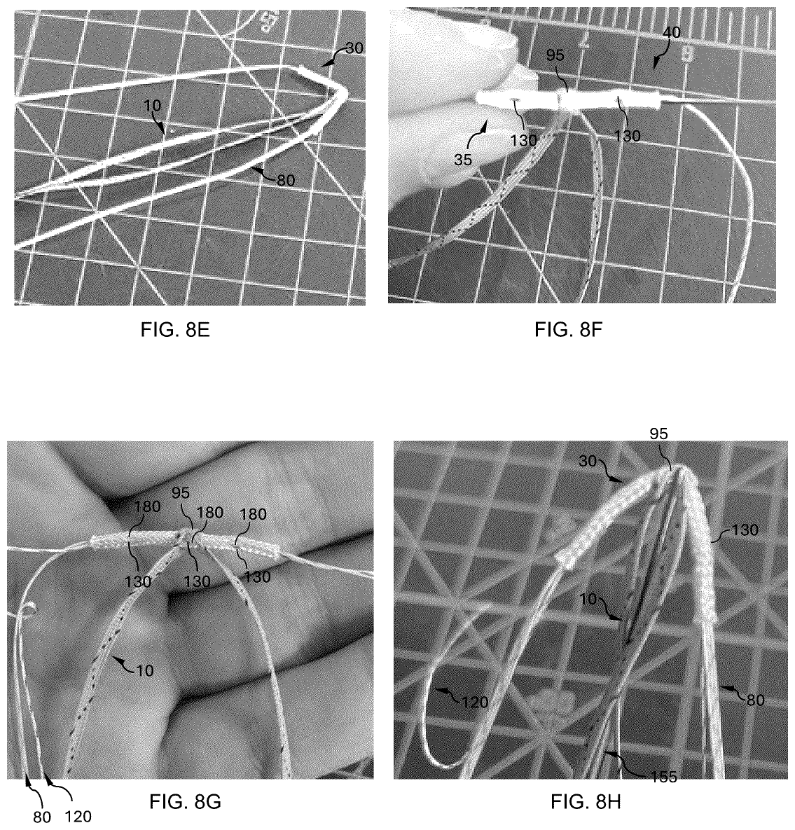

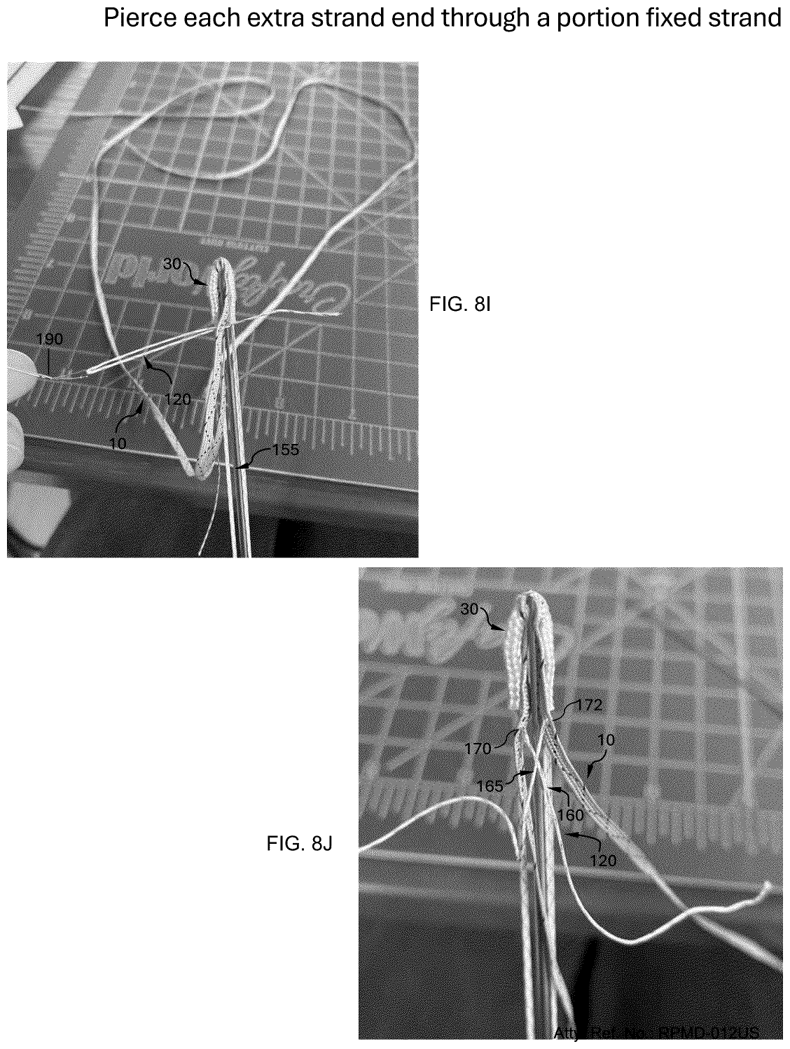

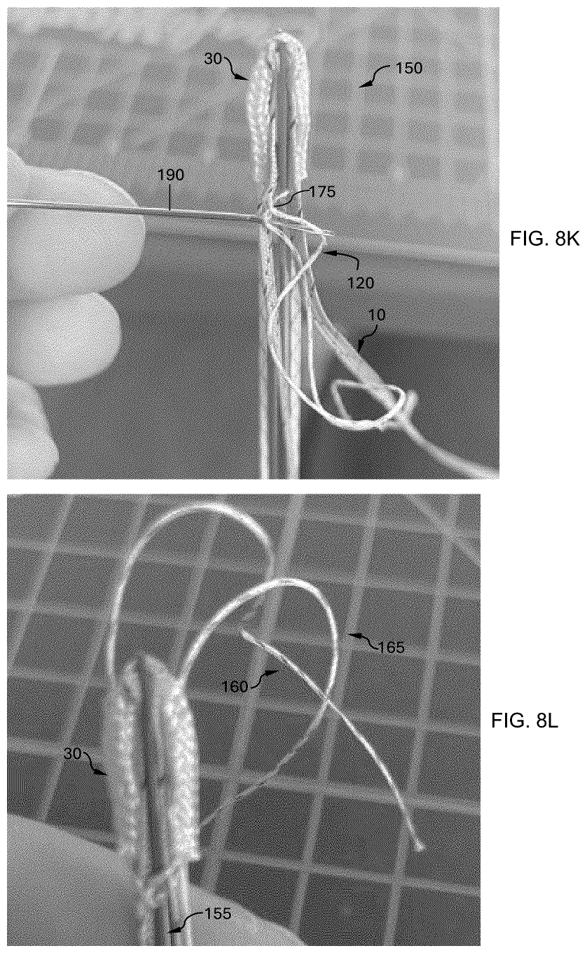

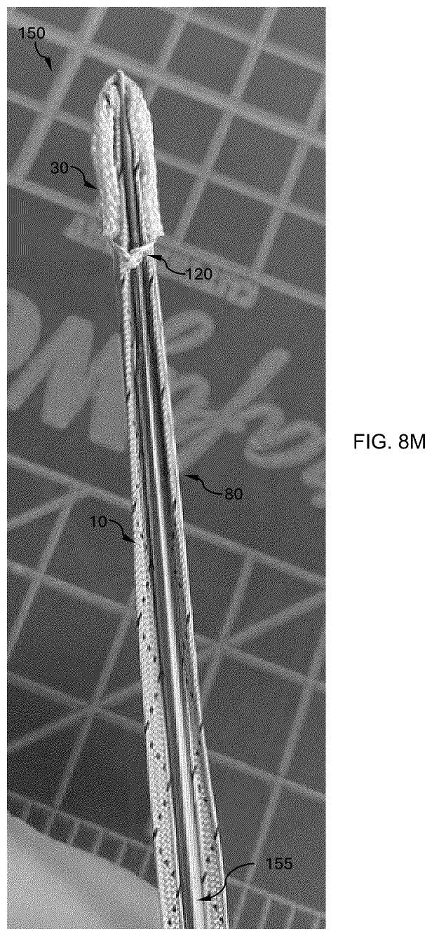

FIGS. 6A-6B, 7A-7D and 8A-8M illustrate a fifth embodiment suture anchor system 150 and method to create the fifth embodiment suture anchor system 150. The suture anchor system 150 comprises a first strand 10 and an anchor 30. The suture anchor system 150 may further comprise a second strand 80. The suture anchor system 150 may further comprise a third strand 120. The first strand 10 comprises a first strand longitudinal axis 100, a first free end 15 and a second free end 20, the first strand 10 comprising a plurality of first strand openings or piercings 25, the plurality of first strand openings or piercings 25 positioned between the first free end 15 and the second free end 20 of the first strand. The second strand 80 comprises a first free end 85 and a second free end 90. The third strand 120 comprises a first free end 160 and a second free end 165.

The anchor 30 comprising a tubular anchor body, a first end 35, a second end 40, a lumen 45, and an anchor material. The anchor 30 may further comprise an outer surface 140. The anchor 30 may further comprise a first sidewall (not shown) and a second sidewall (not shown). The lumen 45 extending between the first end 35 and second end 40 of the anchor 30. The anchor 30 being engaged and/or slidably inserted into each of the plurality of first strand openings 25 of the first strand 10 at a set location or position on the anchor 30 (e.g., first strand 10 is dual pierced). The anchor 30 being inserted transverse to the first strand longitudinal axis 100 through each of the plurality of first strand openings 25. The first free end 15 and the second free end 20 of the first strand 10 are tensioned in the same direction to create a locking collar 95.

At least a portion of the second strand 80 is being weaved into and out of a portion of the anchor 30 in a first weave pattern. The weave pattern comprises at least a portion of the second strand 80 extending over the outer surface 140 and through a portion of the lumen 45 of the anchor 30 at one or more set locations to create one or more loop segments 130, the one or more loop segments 130 contact or are adjacent to the outer surface 140 of the anchor 30. The one or more loop segments 130 are aligned, substantially aligned, and/or parallel to the horizontal axis of the anchor 30. In another embodiment, the weave pattern comprises at least a portion of the second strand 80 extending into the lumen 45 and exiting out of a sidewall of the anchor 30 to create one or more loop segments 130. In one exemplary embodiment, the suture anchor system 150 comprises at least three loop segments 130. At least one of the one or more loop segments 130 may be disposed below or under a portion of the locking collar 95 of the first strand 10. The second strand 80 being slidable or movable relative to the anchor 30.

At least a portion of the third strand 120 being weaved into and out of a portion of the anchor 30 in a second weave pattern. The second weave pattern of the third strand 120 is matching or substantially matching the first weave pattern of the second strand 80. The second weave pattern of the third strand 120 is different than the first weave pattern of the second strand 80. Accordingly, the weave pattern for the third strand 120 comprises extending over the outer surface 140 and through a portion of the lumen 45 of the anchor 30 at one or more set locations to create one or more loop segments 180, at least a portion of the one or more loop segments 180 contact or are adjacent to the outer surface 140 of the anchor 30. The one or more loop segments 180 are aligned, substantially aligned, and/or parallel to the horizontal axis of the anchor 30. In another embodiment, the second weave pattern for the third strand 120 comprises at least a portion of the third strand 120 extending into the lumen 45, entering and exiting out of a sidewall of the anchor 30 to create one or more loop segments 180. In one exemplary embodiment, the suture anchor system 150 comprises at least three loop segments 180. At least one of the one or more loop segments 180 may be disposed below or under a portion of the locking collar 95 of the first strand 10.

The first free end 160 of the third strand 120 extending or piercing through a portion of the first free end 15 of the first strand 10 to create a first intersection point 170, and the second free end 160 of the third strand 120 extending or piercing through a portion of the second free end 20 of the first strand 10 to create a second intersection point 172. The first intersecting point 170 and the second intersecting point 172 may comprise a locking intersecting points. The locking intersecting points may include locking splices. The locking splices comprises a double, triple or quadruple Brummel splice. The locking splice may further comprise a back splice, a chain splice, an eye splice and/or any combination thereof.

The first free end 160 of the third strand 120 extending or piercing through a portion of the second free end 165 of the third strand 120 to create a plurality of intersection points 175. The plurality of intersecting points 175 may comprise a locking intersecting point. The locking intersecting points may include locking splices. The locking splices comprise a double, triple or quadruple Brummel splice. The locking splice may further comprise a back splice, a chain splice, an eye splice and/or any combination thereof. The first free end 160 and the second free end 165 of the third strand 120 being inserted into the lumen 45 of anchor 30. Accordingly, the first free end 160 of the third strand 120 being inserted into the lumen 45 of the anchor at a first end 35 and the second free end 165 of the third strand 120 being inserted into the lumen at the second end 40. Portions of the first free end 160 and the second free end 165 of the third strand 120 may be trimmed. The third strand 120 may be locked relative to the anchor and/or the first strand 10.

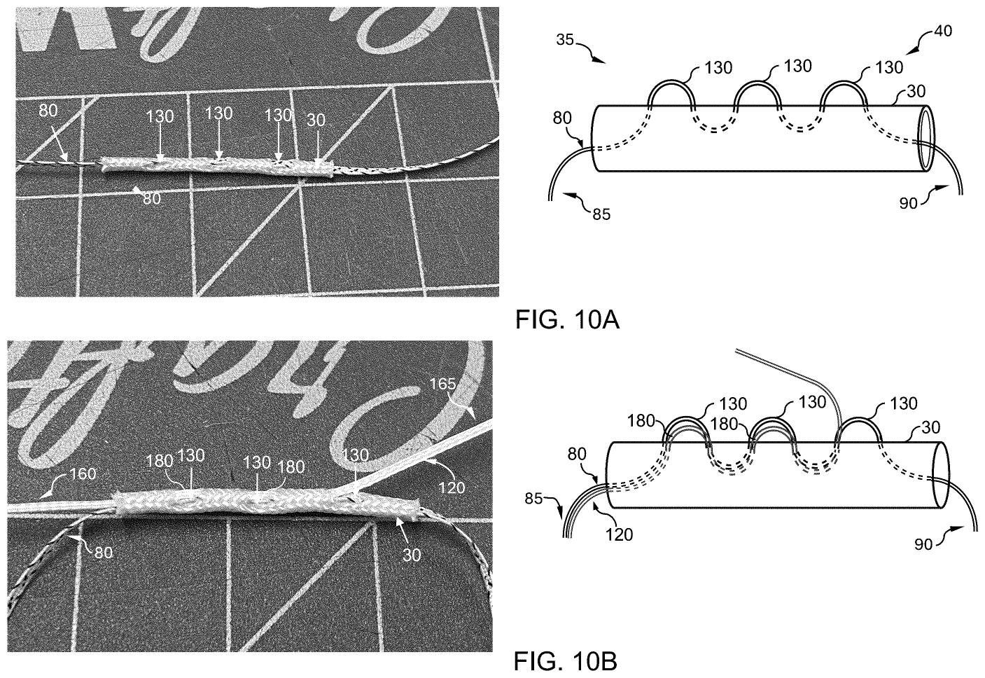

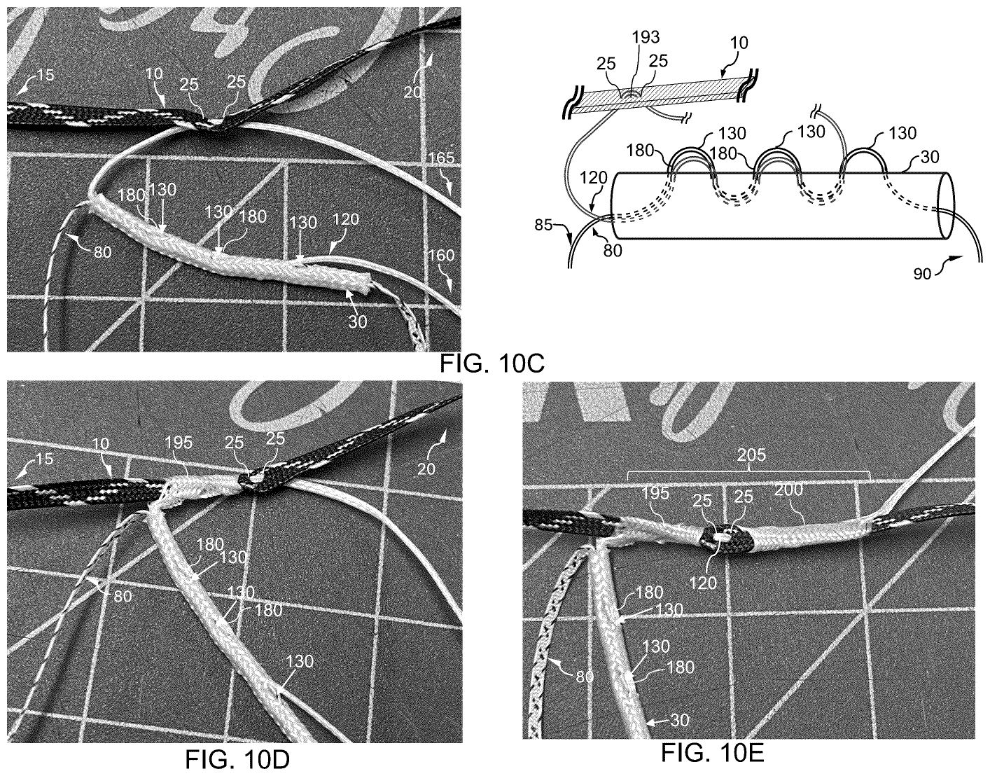

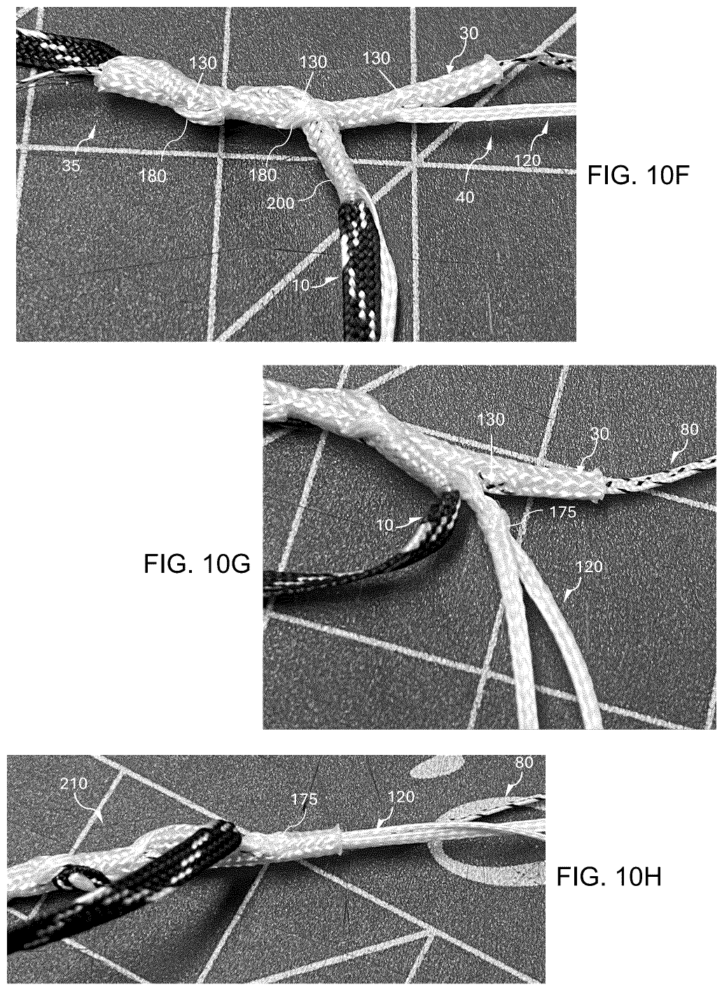

FIGS. 10A-10H depict a sixth embodiment suture anchor system 210 and a method of making the sixth embodiment of a suture anchor system 210. The suture anchor system 210 comprises a first strand 10 and an anchor 30. The suture anchor system 210 may further comprise a second strand 80. The suture anchor system 210 may further comprise a third strand 120. The first strand 10 comprises a first strand longitudinal axis 100, a first free end 15 and a second free end 20. The second strand 80 comprises a first free end 85 and a second free end 90. The third strand 120 comprises a first free end 160 and a second free end 165.

The anchor 30 comprising a tubular anchor body, a first end 35, a second end 40, a lumen 45, and an anchor material. The anchor 30 may further comprise an outer surface 140. The anchor 30 may further comprise a first sidewall (not shown) and a second sidewall (not shown). The lumen 45 extending between the first end 35 and second end 40 of the anchor 30.

At least a portion of the second strand 80 is being weaved into and out of a portion of the anchor 30 in a first weave pattern. The first weave pattern comprises at least a portion of the second strand 80 extending over the outer surface 140 and through a portion of the lumen 45 of the anchor 30 at one or more set locations to create one or more loop segments 130, the one or more loop segments 130 contact or are adjacent to the outer surface 140 of the anchor 30. The one or more loop segments 130 are aligned, substantially aligned, and/or parallel to the horizontal axis of the anchor 30. In another embodiment, the first weave pattern comprises at least a portion of the second strand 80 extending into the lumen 45 and exiting out of a sidewall of the anchor 30 to create one or more loop segments 130. In one exemplary embodiment, the suture anchor system 210 comprises at least three loop segments 130. The second strand 80 being slidable or movable relative to the anchor 30.

At least a portion of the third strand 120 being weaved into and out of a portion of the anchor 30 in a second weave pattern. The second weave pattern of the third strand 120 matches or substantially matches the first weave pattern of the second strand 80. The second weave pattern of the third strand 120 is different than the first weave pattern of the second strand 80. Accordingly, the second weave pattern for the third strand 120 comprises extending over the outer surface 140 and through a portion of the lumen 45 of the anchor 30 at one or more set locations to create one or more loop segments 180, at least a portion of the one or more loop segments 180 contact or are adjacent to the outer surface 140 of the anchor 30. The one or more loop segments 180 are aligned, substantially aligned, and/or parallel to the horizontal axis of the anchor 30. In another embodiment, the second weave pattern for the third strand 120 comprises at least a portion of the third strand 120 extending into the lumen 45, entering and exiting out of a sidewall of the anchor 30 to create one or more loop segments 180. In one exemplary embodiment, the suture anchor system 210 comprises at least two loop segments 180.

At least a portion of the third strand 120 and/or a portion of the first free end 160 of the third strand 120 pierces and/or extends through a portion of the first strand 10 at a first location to create an external segment 193. Alternatively, at least a portion of the third strand 120 and/or a portion of the first free end 160 pierces and/or extends through a plurality of first strand openings 25 at a first location on the first strand 10 to create an at least one external segment 193. The first location between the first free end 15 and the second free end 20 of the first strand 10 includes the at least one external segment 193.

At least a portion of the first strand 10 and/or a second free end 20 of the first strand pierces or extends along the longitudinal axis of a portion of the third strand 120 at a first splice position to create a first splice 195 and pierces or extends along the longitudinal axis of a portion of the third strand 120 at a second splice position to create a second splice 200. The first splice position is spaced apart from the second splice position. The at least one external segment 193 is between the first splice 195 and the second splice 200. The splice region 205 includes the first splice 195 and the second splice 200.

The spliced region 200 encircles a portion of the outer surface 140 of the anchor 30 to extend through at least one of the one or more loop segments 130, 180. The spliced region 200 encircles a portion of the outer surface 140 of the anchor 30 to extend through and under or below the at least one of the one or more loop segments 130, 180 created by the second strand 80 and the third strand 120. Alternatively, the spliced region 200 encircles a portion of the outer surface 140 of the anchor 30 to extend through at least one of the one or more loop segments 130 created by the second strand 80 and/or at least one of the one or more loop segments 180 created by the third strand 120. The spliced region 200 encircles a portion of the outer surface 140 of the anchor 30 to extend through and under or below at least one of the one or more loop segments 130 created by the second strand 80 and/or at least one of the one or more loop segments 180 created by the third strand 120. The encircling may comprise at least half a rotation or greater around the outer surface 140 of the anchor.

The first free end 160 of the third strand 120 extending or piercing through a portion of the second free end 165 of the third strand 10 to create a plurality of intersection points 175. The plurality of intersecting points 175 may comprise a locking intersecting point. The locking intersecting points may include locking splices. The locking splices comprise a double, triple or quadruple Brummel splice. The locking splice may further comprise a back splice, a chain splice, an eye splice and/or any combination thereof. The intersected or locked first free end 160 and the second free end 165 of the third strand piercing and/or extending through the outer surface 140 of the anchor to exit out the lumen 45 at the second end 40. Any remaining portions may be trimmed.

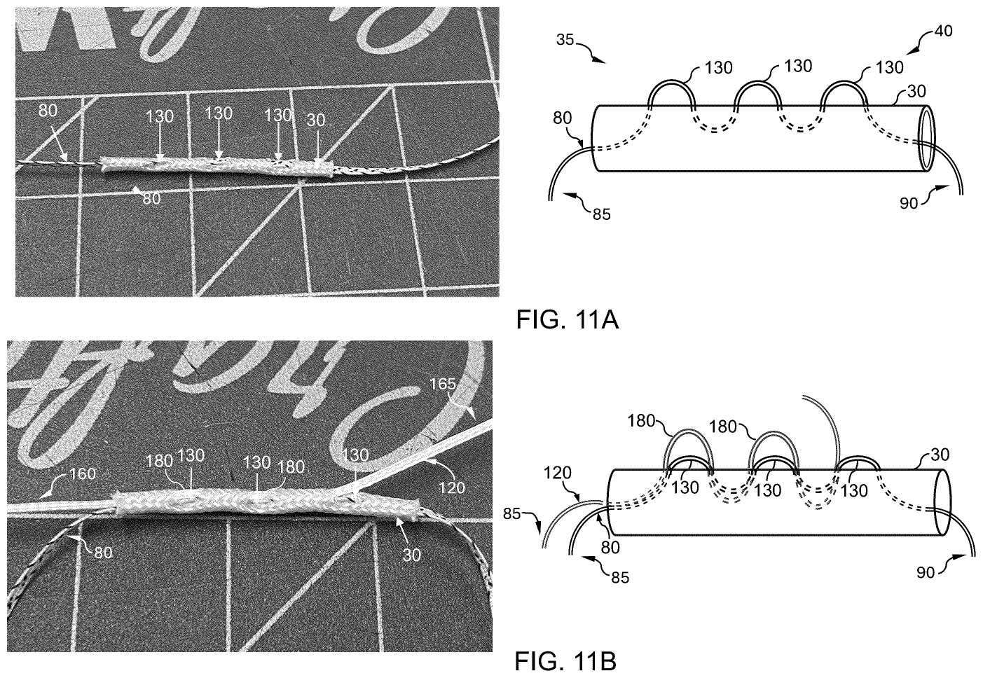

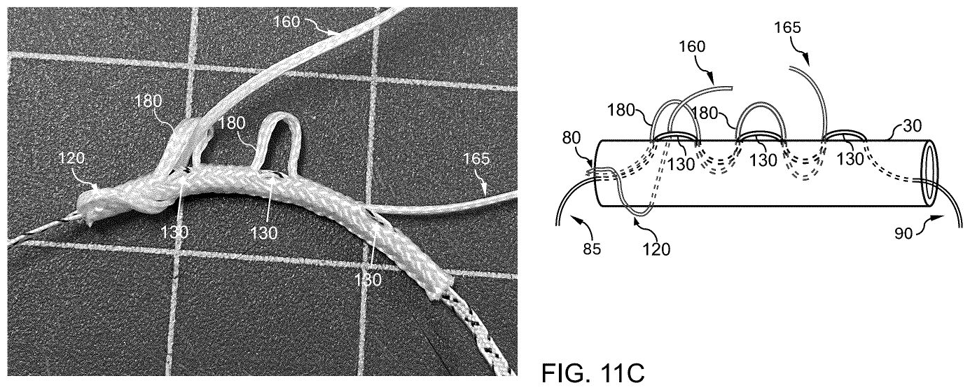

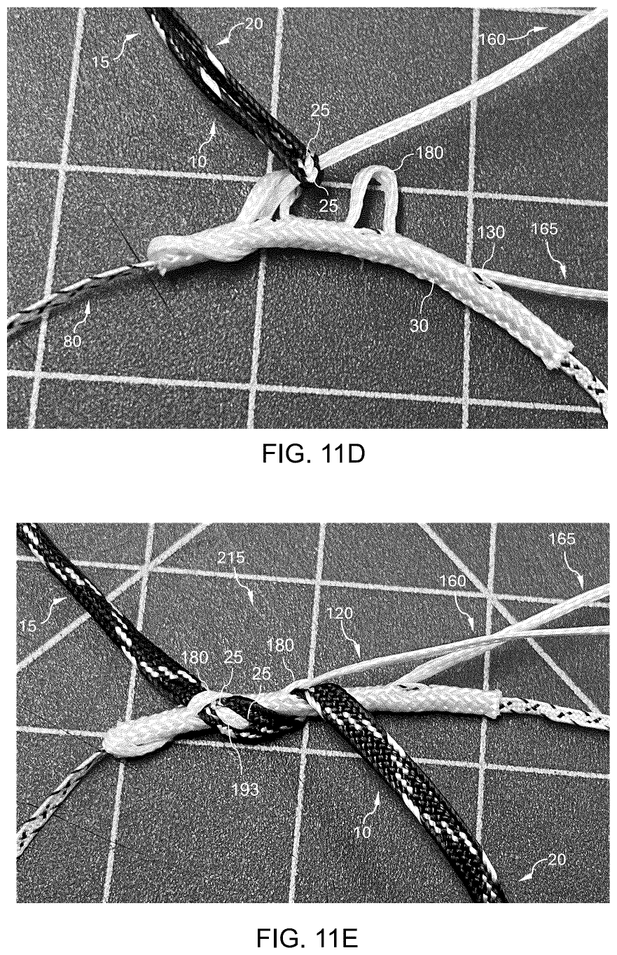

FIGS. 11A-11E depicts a seventh suture anchor system 215 and method of making the seventh embodiment of the suture anchor system 215. The suture anchor system 215 comprises a first strand 10 and an anchor 30. The suture anchor system 215 may further comprise a second strand 80. The suture anchor system 215 may further comprise a third strand 120. The first strand 10 comprises a first strand longitudinal axis 100, a first free end 15 and a second free end 20. The second strand 80 comprises a first free end 85 and a second free end 90. The third strand 120 comprises a first free end 160 and a second free end 165.

The anchor 30 comprising a tubular anchor body, a first end 35, a second end 40, a lumen 45, and an anchor material. The anchor 30 may further comprise an outer surface 140. The anchor 30 may further comprise a first sidewall (not shown) and a second sidewall (not shown). The lumen 45 extending between the first end 35 and second end 40 of the anchor 30.

At least a portion of the second strand 80 is being weaved into and out of a portion of the anchor 30 in a first weave pattern. The first weave pattern comprises at least a portion of the second strand 80 extending over the outer surface 140 and through a portion of the lumen 45 of the anchor 30 at one or more set locations to create one or more loop segments 130, the one or more loop segments 130 contact or are adjacent to the outer surface 140 of the anchor 30. The one or more loop segments 130 are aligned, substantially aligned, and/or parallel to the horizontal axis of the anchor 30. In another embodiment, the first weave pattern comprises at least a portion of the second strand 80 extending into the lumen 45 and exiting out of a sidewall of the anchor 30 to create one or more loop segments 130. In one exemplary embodiment, the suture anchor system 215 comprises at least three loop segments 130. The second strand 80 being slidable or movable relative to the anchor 30.

At least a portion of the third strand 120 being weaved into and out of a portion of the anchor 30 in a second weave pattern. The second weave pattern of the third strand 120 matches or substantially matches the first weave pattern of the second strand 80. The second weave pattern of the third strand 120 is different than the first weave pattern of the second strand 80. Accordingly, the second weave pattern for the third strand 120 comprises extending over the outer surface 140 and through a portion of the lumen 45 of the anchor 30 at one or more set locations to create one or more loop segments 180, at least a portion of the one or more loop segments 180 contact or are adjacent to the outer surface 140 of the anchor 30. The one or more loop segments 180 are aligned, substantially aligned, and/or parallel to the horizontal axis of the anchor 30. In another embodiment, the second weave pattern for the third strand 120 comprises at least a portion of the third strand 120 extending into the lumen 45, entering and exiting out of a sidewall of the anchor 30 to create one or more loop segments 180. In one exemplary embodiment, the suture anchor system 215 comprises at least two loop segments 180.

At least a portion of the first free end 160 of the third strand 120 encircles in a first encircling pattern a portion of the outer surface 140 of the anchor 30 and extends between at least one of the one or more loop segments 130 of the second strand 80 and the one or more loop segments 180 of the third strand 120. At least a portion of the first free end 160 of the third strand 120 encircles in a first encircling pattern a portion of the outer surface 140 of the anchor 30 and extends over at least one of the one or more loop segments 130 of the second strand 80. At least a portion of the first free end 160 of the third strand 120 encircles in a first encircling pattern a portion of the outer surface 140 of the anchor 30 and extends under at least one of the one or more loop segments 180 of the third strand 120. Alternatively, at least a portion of the first free end 160 of the third strand 120 encircles in a first encircling pattern a portion of the outer surface 140 of the anchor 30 and extends between each of the one or more loop segments 130 of the second strand 80 and the one or more loop segments 180 of the third strand 120. At least a portion of the first free end 160 of the third strand 120 encircles in a first encircling pattern a portion of the outer surface 140 of the anchor 30 and extends over each of the one or more loop segments 130 of the second strand 80. At least a portion of the first free end 160 of the third strand 120 encircles in an encircling pattern a portion of the outer surface 140 of the anchor 30 and extends under each of the one or more loop segments 180 of the third strand 120

At least a portion of the third strand 120 and/or a portion of the first free end 160 of the third strand 120 pierces and/or extends through a portion of the first strand 10 at a first location to create an external segment 193. Alternatively, at least a portion of the third strand 120 and/or a portion of the first free end 160 pierces and/or extends through a plurality of first strand openings 25 at a first location on the first strand 10 to create an at least one external segment 193. The first location between the first free end 15 and the second free end 20 of the first strand 10 includes the at least one external segment 193.

At least a portion of the first strand 10, the first free end 15 and/or the second free end 20 of the first strand 10 encircles in a second encircling pattern a portion of the outer surface 140 of the anchor 30 and extends between at least one of the one or more loop segments 130 of the second strand 80 and the one or more loop segments 180 of the third strand 120. At least a portion of the first strand 10, the first free end 15 and/or the second free end 20 of the first strand 10 encircles in a second encircling pattern a portion of the outer surface 140 of the anchor 30 and extends above or over at least one of the one or more loop segments 130 of the second strand 80. At least a portion of the first strand 10, the first free end 15 and/or the second free end 20 of the first strand 10 encircles in a second encircling pattern a portion of the outer surface 140 of the anchor 30 and extends under or below at least one of the one or more loop segments 180 of the third strand 120.

Alternatively, at least a portion of the first strand 10, the first free end 15 and/or the second free end 20 of the first strand 10 encircles in a second encircling pattern a portion of the outer surface 140 of the anchor 30 and extends between each of the one or more loop segments 130 of the second strand 80 and the one or more loop segments 180 of the third strand 120. At least a portion of the first strand 10, the first free end 15 and/or the second free end 20 of the first strand 10 encircles in a second encircling pattern a portion of the outer surface 140 of the anchor 30 and extends above or over each of the one or more loop segments 130 of the second strand 80. At least a portion of the first strand 10, the first free end 15 and/or the second free end 20 of the first strand 10 encircles in a second encircling pattern a portion of the outer surface 140 of the anchor 30 and extends under or below each of the one or more loop segments 180 of the third strand 120. The first encircling pattern may match or substantially match the second encircling pattern. The first encircling pattern may comprise a different encircling pattern than the second encircling pattern.

The first free end 160 of the third strand 120 extending or piercing through a portion of the second free end 165 of the third strand 10 to create a plurality of intersection points 175. The plurality of intersecting points 175 may comprise a locking intersecting point. The locking intersecting points may include locking splices or knots. The locking splices comprise a double, triple or quadruple Brummel splice. The locking splice may further comprise a back splice, a chain splice, an eye splice and/or any combination thereof. The intersected or locked first free end 160 and the second free end 165 of the third strand piercing and/or extending through the outer surface 140 of the anchor to exit out the lumen 45 at the second end 40. Any remaining portions may be trimmed.

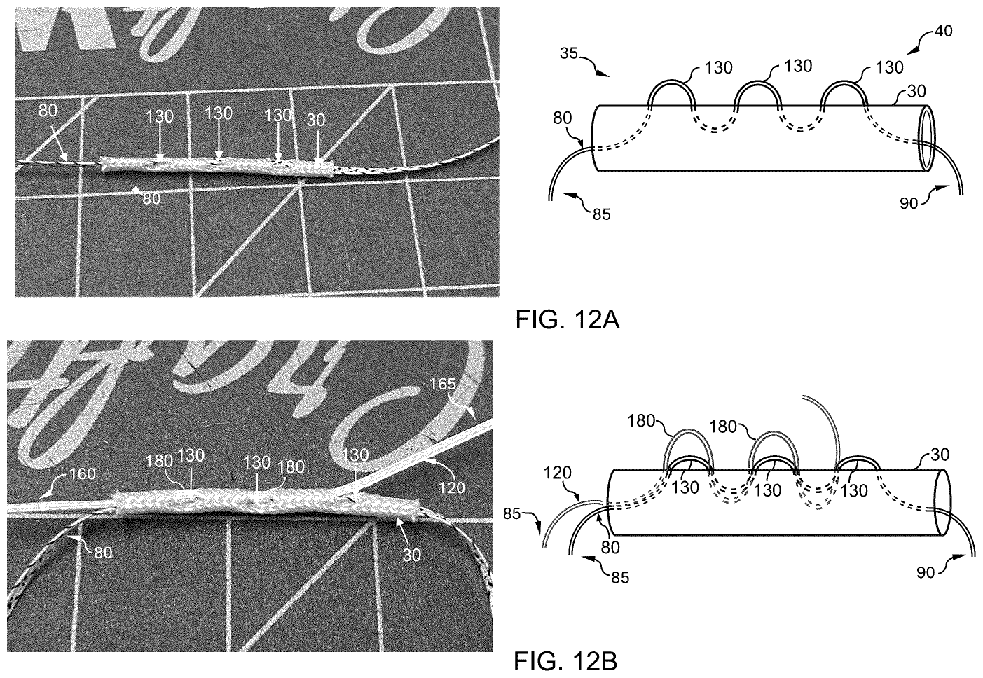

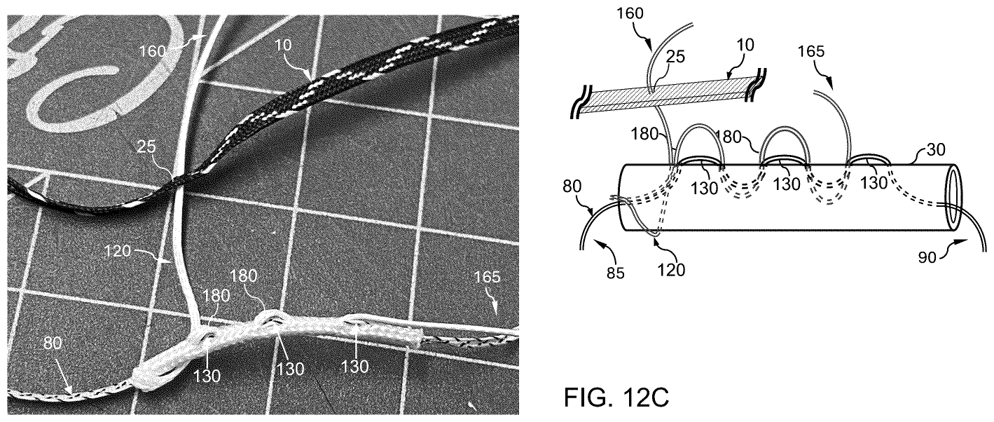

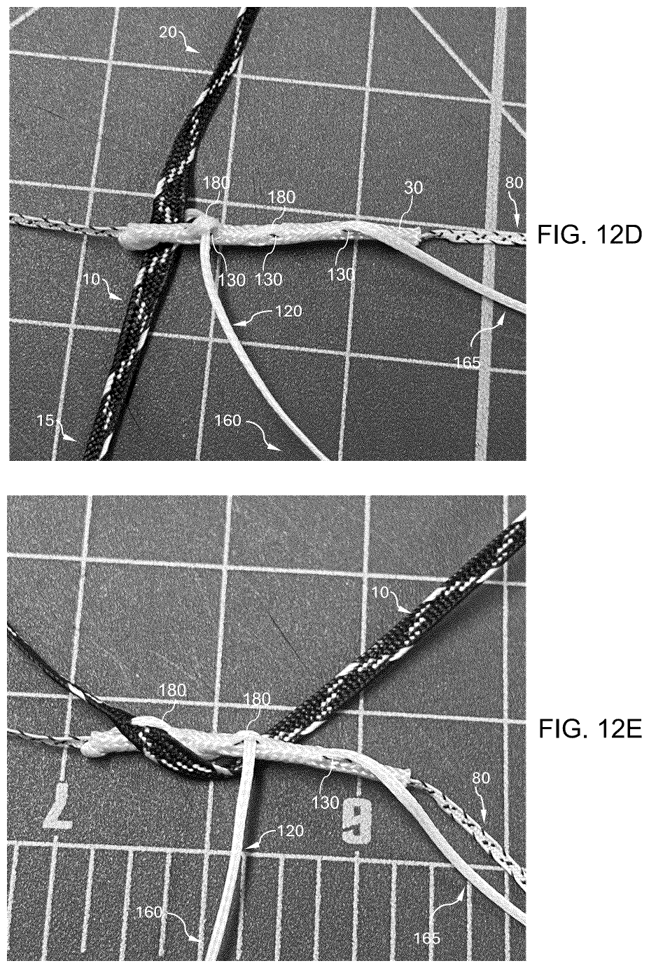

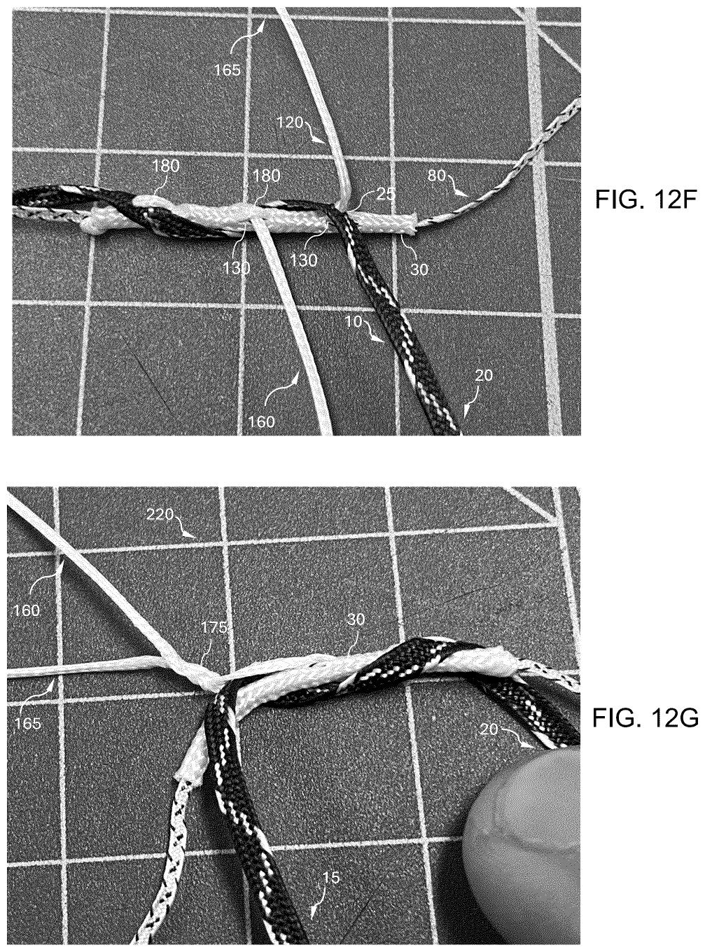

FIGS. 12A-12G depicts an eighth embodiment of a suture anchor system 220 and a method of making an eighth embodiment of a suture anchor system 220. The suture anchor system 220 comprises a first strand 10 and an anchor 30. The suture anchor system 220 may further comprise a second strand 80. The suture anchor system 220 may further comprise a third strand 120. The first strand 10 comprises a first strand longitudinal axis 100, a first free end 15 and a second free end 20. The second strand 80 comprises a first free end 85 and a second free end 90. The third strand 120 comprises a first free end 160 and a second free end 165.

The anchor 30 comprising a tubular anchor body, a first end 35, a second end 40, a lumen 45, and an anchor material. The anchor 30 may further comprise an outer surface 140. The anchor 30 may further comprise a first sidewall (not shown) and a second sidewall (not shown). The lumen 45 extending between the first end 35 and second end 40 of the anchor 30.

At least a portion of the second strand 80 is being weaved into and out of a portion of the anchor 30 in a first weave pattern. The first weave pattern comprises at least a portion of the second strand 80 extending over the outer surface 140 and through a portion of the lumen 45 of the anchor 30 at one or more set locations to create one or more loop segments 130, the one or more loop segments 130 contact or are adjacent to the outer surface 140 of the anchor 30. The one or more loop segments 130 are aligned, substantially aligned, and/or parallel to the horizontal axis of the anchor 30. In another embodiment, the first weave pattern comprises at least a portion of the second strand 80 extending into the lumen 45 and exiting out of a sidewall of the anchor 30 to create one or more loop segments 130. In one exemplary embodiment, the suture anchor system 220 comprises at least three loop segments 130. The second strand 80 being slidable or movable relative to the anchor 30. The one or more loop segments 130 extend outside the outer surface 140 of the anchor 30.

At least a portion of the third strand 120 being weaved into and out of a portion of the anchor 30 in a second weave pattern. The second weave pattern of the third strand 120 matches or substantially matches the first weave pattern of the second strand 80. The second weave pattern of the third strand 120 is different than the first weave pattern of the second strand 80. Accordingly, the second weave pattern for the third strand 120 comprises extending over the outer surface 140 and through a portion of the lumen 45 of the anchor 30 at one or more set locations to create one or more loop segments 180, at least a portion of the one or more loop segments 180 contact or are adjacent to the outer surface 140 of the anchor 30. The one or more loop segments 180 are aligned, substantially aligned, and/or parallel to the horizontal axis of the anchor 30. In another embodiment, the second weave pattern for the third strand 120 comprises at least a portion of the third strand 120 extending into the lumen 45, entering and exiting out of a sidewall of the anchor 30 to create one or more loop segments 180. In one exemplary embodiment, the suture anchor system 220 comprises at least two loop segments 180. The one or more loop segments 180 extend outside the outer surface 140 of the anchor 30.

At least a portion of the first free end 160 of the third strand 120 encircles in a first encircling pattern a portion of the outer surface 140 of the anchor 30 and extends between at least one of the one or more loop segments 130 of the second strand 80 and the one or more loop segments 180 of the third strand 120. At least a portion of the first free end 160 of the third strand 120 encircles in a first encircling pattern a portion of the outer surface 140 of the anchor 30 and extends over at least one of the one or more loop segments 130 of the second strand 80. At least a portion of the first free end 160 of the third strand 120 encircles in a first encircling pattern a portion of the outer surface 140 of the anchor 30 and extends under or below at least one of the one or more loop segments 180 of the third strand 120. Alternatively, at least a portion of the first free end 160 of the third strand 120 encircles in a first encircling pattern a portion of the outer surface 140 of the anchor 30 and extends between each of the one or more loop segments 130 of the second strand 80 and the one or more loop segments 180 of the third strand 120. At least a portion of the first free end 160 of the third strand 120 encircles in a first encircling pattern a portion of the outer surface 140 of the anchor 30 and extends over each of the one or more loop segments 130 of the second strand 80. At least a portion of the first free end 160 of the third strand 120 encircles in a first encircling pattern a portion of the outer surface 140 of the anchor 30 and extends under or below each of the one or more loop segments 180 of the third strand 120. The first encircling pattern may comprise at least half a rotation around the outer surface 140 of the anchor or greater.

At least a portion of the third strand 120 and/or a portion of the first free end 160 of the third strand 120 pierces and/or extends through a portion of the first strand 10 at a first location and at a second location. Alternatively, at least a portion of the third strand 120 and/or a portion of the first free end 160 pierces and/or extends through the first strand opening 25 at a first location on the first strand 10 and a second strand opening 25 at a second location.

At least a portion of the first strand 10, the first free end 15 and/or the second free end 20 of the first strand 10 encircles in a second encircling pattern a portion of the outer surface 140 of the anchor 30 and extends between at least one of the one or more loop segments 130 of the second strand 80 and the one or more loop segments 180 of the third strand 120. At least a portion of the first strand 10, the first free end 15 and/or the second free end 20 of the first strand 10 encircles in a second encircling pattern a portion of the outer surface 140 of the anchor 30 and extends above or over at least one of the one or more loop segments 130 of the second strand 80. At least a portion of the first strand 10, the first free end 15 and/or the second free end 20 of the first strand 10 encircles in a second encircling pattern a portion of the outer surface 140 of the anchor 30 and extends under or below at least one of the one or more loop segments 180 of the third strand 120. The first encircling pattern may comprise at least half a rotation around the outer surface 140 of the anchor or greater.

Alternatively, at least a portion of the first strand 10, the first free end 15 and/or the second free end 20 of the first strand 10 encircles in a second encircling pattern a portion of the outer surface 140 of the anchor 30 and extends between each of the one or more loop segments 130 of the second strand 80 and the one or more loop segments 180 of the third strand 120. At least a portion of the first strand 10, the first free end 15 and/or the second free end 20 of the first strand 10 encircles in a second encircling pattern a portion of the outer surface 140 of the anchor 30 and extends above or over each of the one or more loop segments 130 of the second strand 80. At least a portion of the first strand 10, the first free end 15 and/or the second free end 20 of the first strand 10 encircles in a second encircling pattern a portion of the outer surface 140 of the anchor 30 and extends under or below each of the one or more loop segments 180 of the third strand 120. The first encircling pattern may match or substantially match the second encircling pattern. The first encircling pattern may comprise a different encircling pattern than the second encircling pattern.

In another embodiment, at least a portion of the first strand 10, the first free end 15 and/or the second free end 20 of the first strand 10 encircles in a second encircling pattern a portion of the outer surface 140 of the anchor 30 and extends under or below each of the one or more loop segments 130 of the second strand 80 and the one or more loop segments 180 of the third strand 120. At least a portion of the first strand 10, the first free end 15 and/or the second free end 20 of the first strand 10 encircles in a second encircling pattern a portion of the outer surface 140 of the anchor 30 and extends under or below both of the one or more loop segments 130 of the second strand 80 and the one or more loop segments 180 of the third strand 120. At least a portion of the first strand 10, the first free end 15 and/or the second free end 20 of the first strand 10 may contact a portion of the outer surface 140 of the anchor 30.

The first free end 160 of the third strand 120 extending or piercing through a portion of the second free end 165 of the third strand 120 to create a plurality of intersection points 175. The plurality of intersecting points 175 may comprise a locking intersecting point. The locking intersecting points may include locking splices or knots. The locking splices comprise a double, triple or quadruple Brummel splice. The locking splice may further comprise a back splice, a chain splice, an eye splice and/or any combination thereof. The intersected or locked first free end 160 and the second free end 165 of the third strand piercing and/or extending through the outer surface 140 of the anchor to exit out the lumen 45 at the second end 40. Any remaining portions may be trimmed.

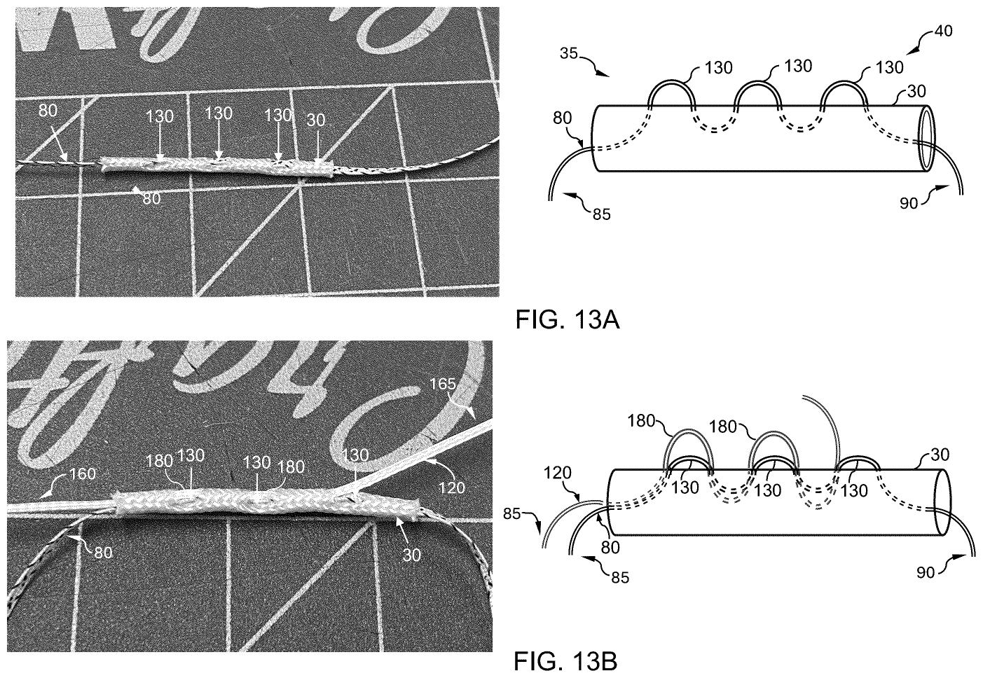

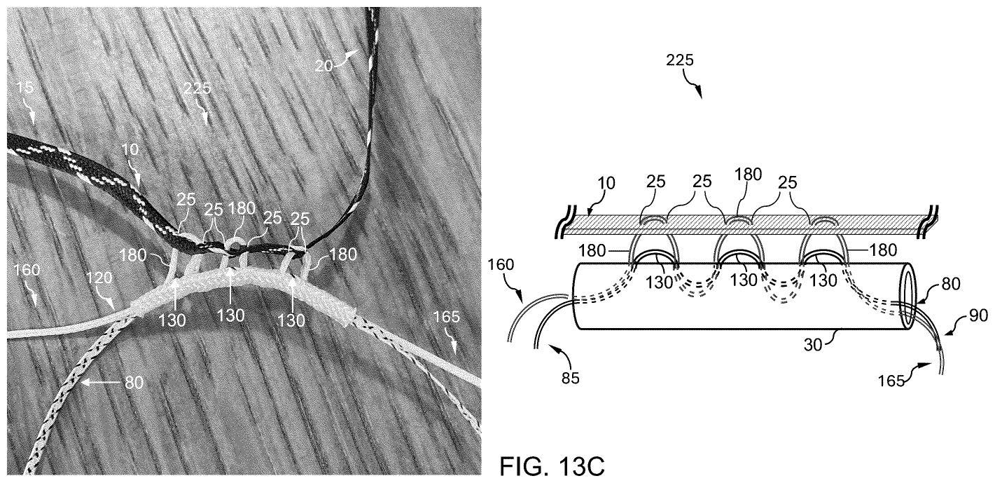

FIGS. 13A-13C depicts a ninth embodiment of a suture anchor system 225 and a method of making a ninth embodiment of a suture anchor system 225. The suture anchor system 225 comprises a first strand 10 and an anchor 30. The suture anchor system 225 may further comprise a second strand 80. The suture anchor system 225 may further comprise a third strand 120. The first strand 10 comprises a first strand longitudinal axis 100, a first free end 15 and a second free end 20. The second strand 80 comprises a first free end 85 and a second free end 90. The third strand 120 comprises a first free end 160 and a second free end 165.

The anchor 30 comprising a tubular anchor body, a first end 35, a second end 40, a lumen 45, and an anchor material. The anchor 30 may further comprise an outer surface 140. The anchor 30 may further comprise a first sidewall (not shown) and a second sidewall (not shown). The lumen 45 extending between the first end 35 and second end 40 of the anchor 30.

At least a portion of the second strand 80 is being weaved into and out of a portion of the anchor 30 in a first weave pattern. The first weave pattern comprises at least a portion of the second strand 80 extending over the outer surface 140 and through a portion of the lumen 45 of the anchor 30 at one or more set locations to create one or more loop segments 130, the one or more loop segments 130 contact or are adjacent to the outer surface 140 of the anchor 30. The one or more loop segments 130 are aligned, substantially aligned, and/or parallel to the horizontal axis of the anchor 30. In another embodiment, the first weave pattern comprises at least a portion of the second strand 80 extending into the lumen 45 and exiting out of a sidewall of the anchor 30 to create one or more loop segments 130. In one exemplary embodiment, the suture anchor system 220 comprises at least three loop segments 130. The second strand 80 being slidable or movable relative to the anchor 30. The one or more loop segments 130 extend outside the outer surface 140 of the anchor 30.

At least a portion of the third strand 120 being weaved into and out of a portion of the anchor 30 and a portion of the first strand 10 in a second weave pattern. The second weave pattern of the third strand 120 matches or substantially matches the first weave pattern of the second strand 80. The second weave pattern of the third strand 120 is different than the first weave pattern of the second strand 80. Accordingly, the second weave pattern for the third strand 120 comprises extending over the outer surface 140 and through a portion of the lumen 45 of the anchor 30 and a portion of the first strand at one or more set locations to create one or more loop segments 180, at least a portion of the one or more loop segments 180 contact or are adjacent to the outer surface of the first strand 10 and/or the outer surface 140 of the anchor 30. The one or more loop segments 180 are aligned, substantially aligned, and/or parallel to the horizontal axis of the anchor 30. In another embodiment, the second weave pattern for the third strand 120 comprises at least a portion of the third strand 120 extending into the lumen 45, entering to exiting out of a sidewall of the anchor 30 and pierce a portion of the first strand 10 at at least two locations to create one or more loop segments 180. In one exemplary embodiment, the suture anchor system 225 comprises at least two or more loop segments 180 or at least three loop segments. The one or more loop segments 180 extend outside the outer surface 140 of the anchor 140 and/or the outer surface of the first strand 10.

In another embodiment, a first free end 160 of the third strand 120 passes through the outer surface 140 or a sidewall of the anchor 30 and passes or extends through a portion of the first strand 10 into at least a portion of the lumen 45 of the anchor 45 to create one or more loop segments 180 extending outside the outer surface of the first strand 10. At least a portion of the first strand 10, the first free end 15 and/or the second free end 20 of the first strand 10 encircles a portion of the outer surface 140 of the anchor 30. The encircling may comprise at least a half a rotation around the outer surface 140 of the anchor or greater.

The first free end 160 of the third strand 120 extending or piercing through a portion of the second free end 165 of the third strand 120 to create a plurality of intersection points 175. The plurality of intersecting points 175 may comprise a locking intersecting point. The locking intersecting points may include locking splices or knots. The locking splices comprise a double, triple or quadruple Brummel splice. The locking splice may further comprise a back splice, a chain splice, an eye splice and/or any combination thereof. Any remaining portions may be trimmed.

The first strand 10 is capable of being locked relative to the anchor 30. Alternatively, the first or fixed strand 10 can be non-sliding or substantially non-sliding relative to the anchor. The locking of the first or fixed strand 10 to the anchor 30 prevents the first or fixed strand 10 from changing or substantially changing its positioning relative to the anchor 30. Locking the first or fixed strand 10 to the anchor 30 improves the ability to tension the first or fixed strand 10, such as during bridging techniques. In one embodiment, the first or fixed strand 10 is locked relative to the anchor 30 by a cinch lock, a wedge lock, a compressive lock and/or a friction lock. Furthermore, the second strand 80 or third strand 120 may be slidable or movable relative to the anchor 30. Accordingly, the third strand 120 may also be fixed or non-movable relative to the anchor 30, and may be fixed or non-movable with the first strand 10.

The first strand 10, the second strand 80 and/or the third strand 120 may comprise a flat suture, a round suture, a flat-to-round suture, and/or any combination thereof. The flat configuration and/or the round configuration may comprise a coreless or boreless suture and/or without a lumen or a bore. In one embodiment, the first or fixed strand 10 is a flat suture, the flat suture may comprise FIBERWIRE, TIGERWIRE, FIBERCHAIN AND/OR FIBERTAPE suture.

The anchor 30, the first strand 10, the second strand 80 and/or the third strand 120 may include any soft, flexible material. The flexible material may comprise a textile. The textile includes one or more fibers or filaments to create one or more yarns. The one or more yarns may be intersected in a textile structure to create suture strands or sutures. The textile structure may further comprise a braided, woven or knitted structures. The textile structure may further include monofilament, multifilament and/or pseudo monofilament.

The anchor 30, the first strand 10, the second strand 80 and/or the third strand 120 comprises may comprise a material. The material comprises a polymer or a metal material. The materials may comprise synthetic materials, natural materials and/or any combination thereof. The materials may further comprise biodegradable materials or non-biodegradable materials. The polymer materials may comprise polyamide, polypropylene, polybutester, polyester, nylon, polyethylene terephthalate, polyethylene, ultra-high weight molecular polyethylene, and/or any combination thereof.

### Methods of Creating Suture Anchor Systems

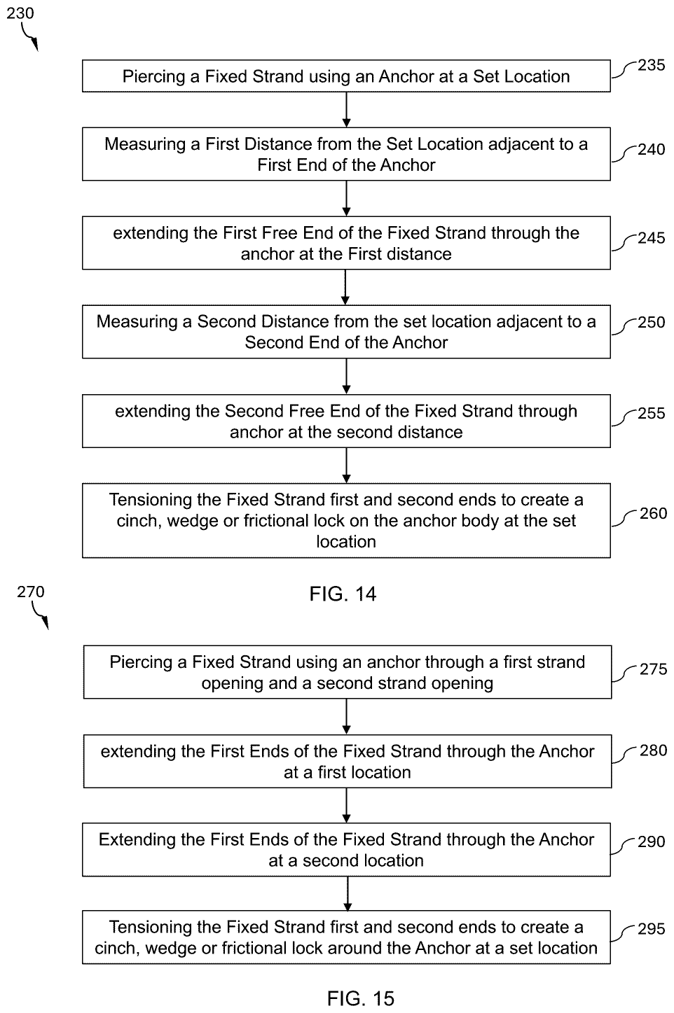

With reference to FIGS. 1A-1D, 2A-2D, 14 and 15, a method 230 of creating a first embodiment suture anchor system 5 comprises the steps of: Piercing a first strand or fixed strand through a first strand opening at a set location 235, the first strand opening being transverse to a longitudinal axis of the first strand; measuring a first distance from the set location adjacent to a first end of the anchor 240; exiting or extending the first free end of the first strand through the anchor at the first distance 245; measuring a second distance from the set location adjacent to a second end of the anchor 250; exiting or extending the second free end of the first strand through the anchor at the second distance 255; and tensioning the first and second ends of the first strand to create a locking collar to fix, cinch or lock the first strand relative to the anchor 260. The first strand opening of the first strand is transverse to the longitudinal axis of the first strand. The first distance comprises a same distance as the second distance. The first distance comprises a different distance as the second distance. The first and/or the second distance comprises 10 mm or less, 8 mm or less, and/or 5 mm or less. The first distance and the second distance are disposed 180 degrees apart.

In another embodiment, the method of creating a suture anchor system 5 comprises the steps of: Piercing a first strand or fixed strand through a first strand opening at a set location 235, the first strand opening being transverse to a longitudinal axis of the first strand; exiting or extending the first free end of the first strand through the anchor at a first location or distance 245; exiting or extending the second free end of the first strand through the anchor at the second distance or location 255; and tensioning the first and second ends of the first strand to create a locking collar to fix, cinch or lock the first strand relative to the anchor 260.

In another embodiment, the method 270 of creating the first embodiment suture anchor system 105 comprises the steps of: piercing a first strand using an anchor through a first strand opening and a second strand opening 275; extending the first end of the first strand through the anchor at a first location adjacent to the first end of the anchor; extending the second end of the first strand through the anchor at a second location adjacent to the second end of the anchor; tensioning and tensioning the first and second ends of the first strand to create a locking collar to fix, cinch or lock the first strand relative to the anchor 295.

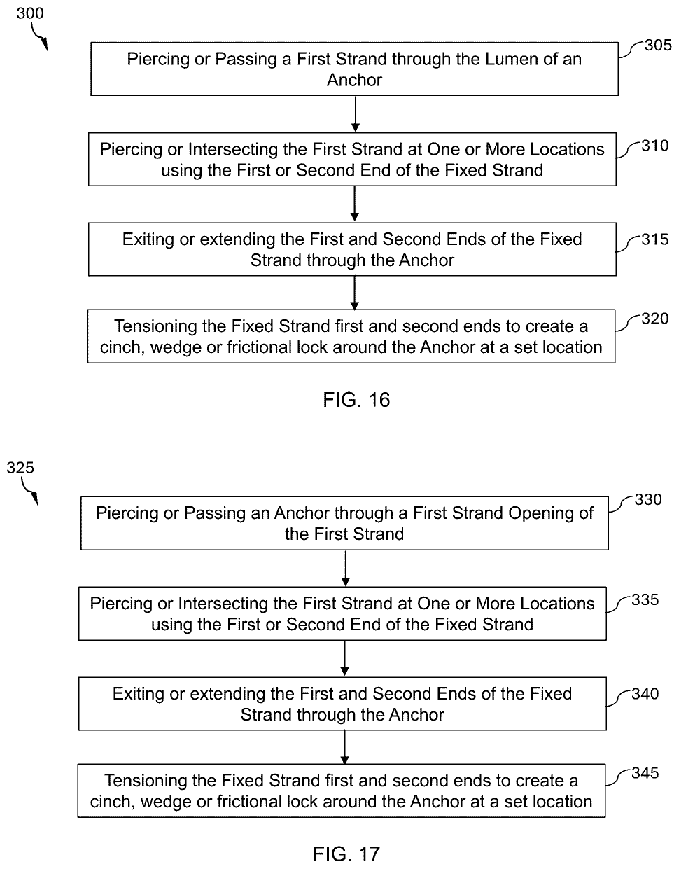

With reference to FIGS. 3A-3C and 16, a method 300 of creating a second embodiment of the suture anchor system 110 comprising the steps of: Passing or extending a portion of the first strand through a portion of a lumen of an anchor 305, the first free end 15 and the second free end 20 extending from the outer surface of the anchor; Piercing or intersecting a first free end and a second free end of the first strand to create one or more intersection regions or points 310, the one or more intersection points may comprise locking one or more intersection points; extending a first free end of the first strand through the anchor at a first position at or near the first end of the anchor and a second free end of the first strand through the anchor at a second position at the second end of the anchor 315; tensioning the first and second end of the first strand to fix, cinch or lock the first strand relative to the anchor to position the one or more intersection regions on the outer surface of the anchor 320. The method of creating a suture anchor assembly may further comprise the step of sliding the at one or more locking intersection regions, points or locations at or adjacent to the outer diameter or outer surface of the anchor. The method 300 may further comprise the steps of: inserting or extending a second strand through the anchor, the second strand may be sliding or movable relative to the anchor. The method 300 may further comprise the steps of inserting or extending a second strand and third strand through the anchor, the second strand and/or third strand may be sliding or movable relative to the anchor.

With reference to FIGS. 4A-4C and 17, a method 325 of creating a third embodiment of the suture anchor system 125 comprising the steps of: Passing or extending a portion of an anchor through a first strand opening of the first strand 330; Piercing or intersecting a first free end and a second free end of the first strand to create one or more intersection regions, the one or more intersection points 335 may comprise locking intersection points; extending a first free end of the first strand through the anchor at a first position at or near the first end of the anchor and a second free end of the first strand through the anchor at a second position at the second end of the anchor 340; tensioning the first and second end of the first strand to fix, cinch or lock the first strand relative to the anchor to position the one or more intersection regions or points on the outer surface of the anchor 345. The method of creating a suture anchor system 125 may further comprise the step of sliding the at one or more locking intersection regions, points or locations at or adjacent to the outer diameter or outer surface of the anchor. The method 300 may further comprise the steps of: inserting or extending a second strand through the anchor, the second strand may be sliding or movable relative to the anchor. The method 300 may further comprise the steps of inserting or extending a second strand and third strand through the anchor, the second strand and/or third strand may be sliding or movable relative to the anchor.

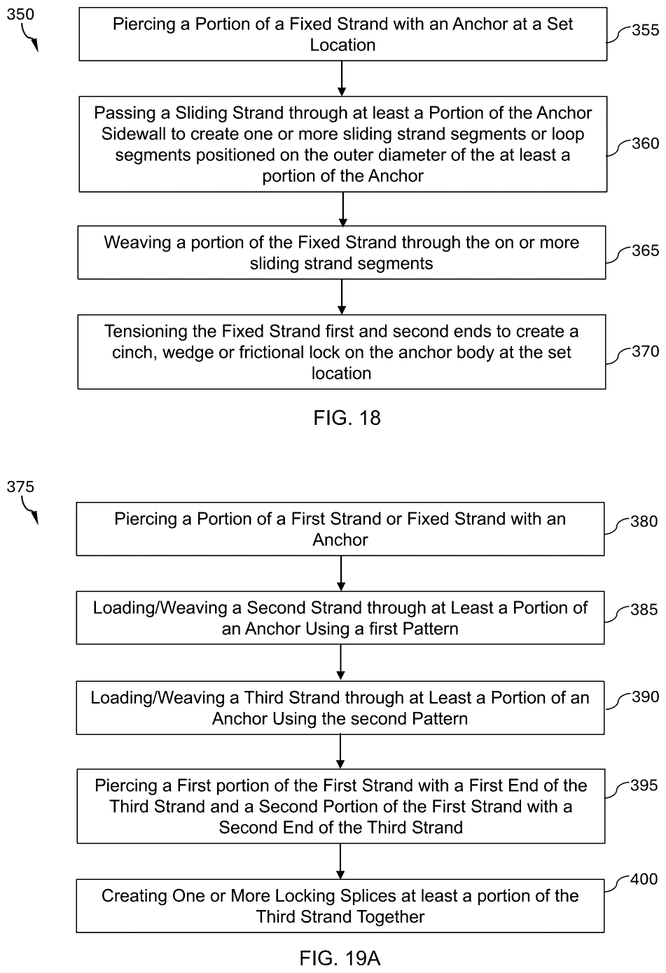

With reference to FIGS. 5A-5D and 18, a method 350 of creating a fourth embodiment suture anchor system 128 comprises the steps of: piercing a portion of a first strand with an anchor at a set location 355; passing a portion of a second strand into at least a portion of the lumen of the anchor to create one or more loop segment extending outside the outer surface the anchor 360, the second strand being slidable relative to the anchor; Weaving a portion of the first strand through or below the one or more loop segments to create one or more locking intersection locations 365; and tensioning the second strand to lock, cinch or wedge the first strand relative to the anchor at the one or more intersection locations 370.

In another embodiment, the method 350 of creating a fourth embodiment suture anchor system comprises the steps of: extending an anchor through a first strand opening of the first strand, the first strand opening positioned between the first and second free ends of the first strand; passing a second strand through a plurality of anchor body openings of the anchor to create at least one loop segment, the at least one loop segment positioned at or adjacent to an outer diameter of the anchor, the second strand being slidable relative to the anchor; weaving the first strand through the at least one loop segment to create at least one locking intersection location; and Tensioning the second strand to lock, cinch or wedge the first strand relative to the anchor at the least one intersection locations.

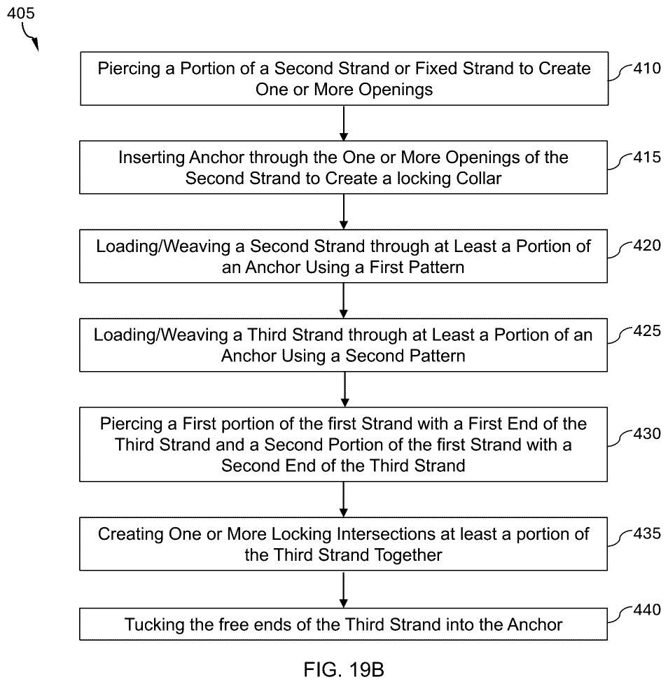

With reference to FIGS. 6A-6B, 7A-7D, 8A-8M, and 19A-19B a method 375 of creating a fifth suture anchor system 150 comprises the steps of: piercing or extending an anchor through a first strand opening and a second strand opening of the first strand to create a first free end and a second free end 380, the first strand opening and second strand opening positioned between a first and second free ends of the first strand; loading or weaving a second strand through an anchor in a first weave pattern 385, the second strand being slidable relative to the anchor; loading or weaving a third strand through the anchor in a second weave pattern to create a first free end and a second free end 390; extending or piercing the first free end of the third strand through a portion of the first free end of the first strand and the second free end of the third strand through a portion of the second free end of the first strand 395; intersecting the first free end of the third strand and the second free end of the third strand at one or more locations to create one or more locking intersections 400. The method 375 may further comprise tucking the first free end of the third strand into a first end of the anchor and the second free end of the third strand into a second end of the anchor. The method may further comprise trimming the first free end and/or the second free end of the third strand. The one or more locking intersections may comprise a double, triple or quadruple Brummel splice. The locking splice making the first strand and the third strand fixed or non-slidable relative to the anchor.

Alternatively, a method 405 of creating the fifth embodiment suture anchor system 150 comprising the steps of: piercing a portion of a first strand to create one or more first strand openings 410; inserting anchor through the one or more first strand openings of the first strand to create a first free end and a second free end 415; loading or weaving a second strand through at least a portion of an anchor in a first weave pattern 420, the second strand being slidable relative to the anchor; loading or weaving a third strand through the anchor in a second weave pattern to create a first free end and a second free end 425; piercing a portion of the first free end of the first strand with a first free end of the third strand and piercing a portion of the second free end of the first strand with a second free end of the third strand 430; intersecting a portion of the first free end and the second free end of the third strand one or more times to create one or more intersecting points, the intersecting points may include locking intersecting points 435; and tucking the first free end of the third strand into a first end of the anchor and the second free end of the third strand into a second end of the anchor 440. The method 405 may further comprise trimming the first free end and/or the second free end of the third strand. The method 405 may further comprise loading the fifth embodiment suture anchor system 150 onto an inserter. The one or more locking splices or intersecting points may comprise a double, triple or quadruple Brummel splice. The locking splice making the first strand fixed or non-slidable relative to the anchor. The first weave pattern may be the same or different than the second weave pattern.

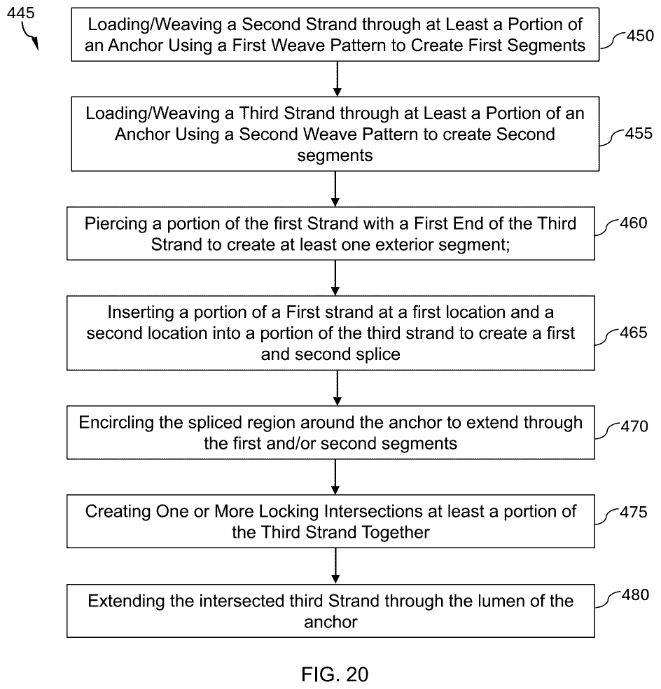

With reference to FIGS. 10A-10H and 20, the method 445 of creating a sixth suture anchor system 210 comprises the steps of: weaving a portion of a second strand through at least a portion of an anchor in a first weave pattern to create one or more first external segments 450, the first one or more first external segments extend from or contact the anchor outer surface; weaving a portion of a third strand through at least a portion of an anchor in a second weave pattern to create one or more second external segments 450, the one or more second external segments extend from or contact the anchor outer surface 455; piercing a portion of the first strand with a first end of the third strand to create at least one strand segment 460; inserting a portion of a first strand at a first location into a portion of a third strand to create a first splice and a portion of a first strand at a second location into a portion of a third strand to create a second splice 465, the first splice is spaced apart from the second splice, the at least one strand segment is positioned between the first splice and the second splice, the splice region includes the first splice and the second splice; encircling the spliced region around the anchor outer surface to extend through the one or more first external segments and/or the one or more second external segments 470; intersecting the first free end and the second free end of the third strand to create one or more intersecting points 475, the one or more intersecting points includes locking one or more intersecting points; and extending the intersecting first free end and second free end of the third strand through the outer surface of the anchor and exiting out the lumen of the anchor 480. The step of encircling the spliced region around the anchor outer surface to extend through the one or more first external segments and/or the one or more second external segments 470 comprises the step of extending between the one or more first external segments and the one or more second external segments and/or extending below the one or more second external segments. The first weave pattern may be the same or different than the second weave pattern. The encircling may comprise at least a one-quarter rotation or greater.

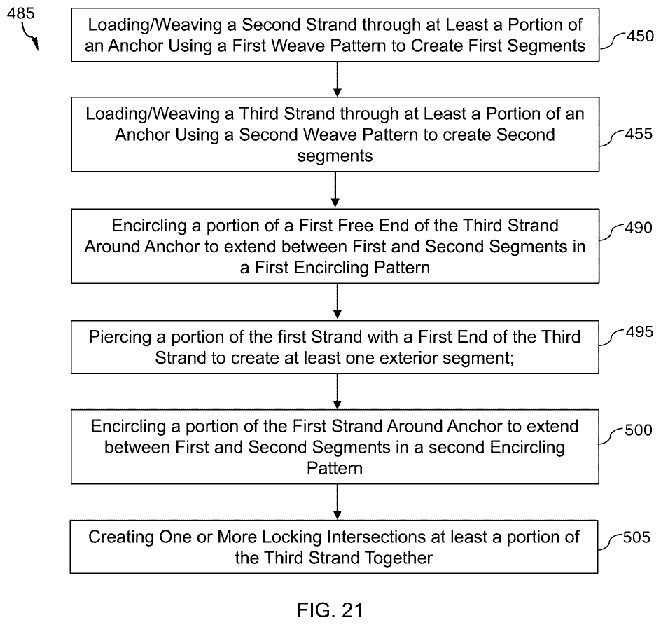

With reference to FIGS. 11A-11E and 21, the method 485 of creating a seventh suture anchor system 215 comprises the steps of: weaving a portion of a second strand through at least a portion of an anchor in a first weave pattern to create one or more first external segments 450, the first one or more first external segments extend from or contact the anchor outer surface; weaving a portion of a third strand through at least a portion of an anchor in a second weave pattern to create one or more second external segments 455, the one or more second external segments extend from or contact the anchor outer surface; encircling a portion of a first free end of a third strand around outer surface of anchor in a first encircling pattern and extending the portion of the first free end between the one or more first external segments and the one or more second external segments 490; piercing a portion of the first strand with a first end of the third strand to create at least one strand segment 495; encircling a portion of the first strand around outer surface of anchor in a second encircling pattern and extending the portion of the first free end between the one or more first external segments and the one or more second external segments 500; and intersecting the first free end and the second free end of the third strand to create one or more intersecting points 505, the one or more intersecting points includes locking one or more intersecting points. The step of encircling around the anchor outer surface to extend through the one or more first external segments and/or the one or more second external segments comprises the step of extending between the one or more first external segments and the one or more second external segments and/or extending below the one or more second external segments. The first weave pattern may be the same or different than the second weave pattern. The first encircling pattern may be the same or different than the second encircling pattern. The encircling pattern may comprise at least a half rotation around the outer surface of the anchor or greater.

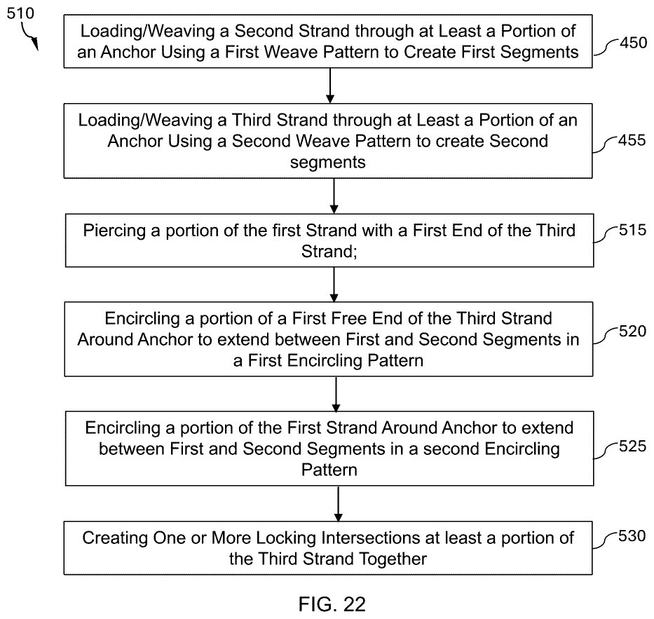

With reference to FIGS. 12A-12G and 22, the method 510 of creating an eighth suture anchor system 220 comprises the steps of: weaving a portion of a second strand through at least a portion of an anchor in a first weave pattern to create one or more first external segments 450, the first one or more first external segments extend from or contact the anchor outer surface; weaving a portion of a third strand through at least a portion of an anchor in a second weave pattern to create one or more second external segments 455, the one or more second external segments extend from or contact the anchor outer surface; piercing a portion of the first strand with a first end of the third strand 515; encircling a portion of a first free end of a third strand around outer surface of anchor in a first encircling pattern and extending the portion of the first free end between the one or more first external segments and the one or more second external segments 520; encircling a portion of the first strand around outer surface of anchor in a second encircling pattern and extending the portion of the first free end between the one or more first external segments and the one or more second external segments 525; and intersecting the first free end and the second free end of the third strand to create one or more intersecting points 530, the one or more intersecting points includes locking one or more intersecting points. The step of encircling around the anchor outer surface to extend through the one or more first external segments and/or the one or more second external segments comprises the step of extending between the one or more first external segments and the one or more second external segments and/or extending below the one or more second external segments. The first weave pattern may be the same or different than the second weave pattern. The first encircling pattern may be the same or different than the second encircling pattern. The encircling pattern may comprise at least a half rotation around the outer surface of the anchor or greater.

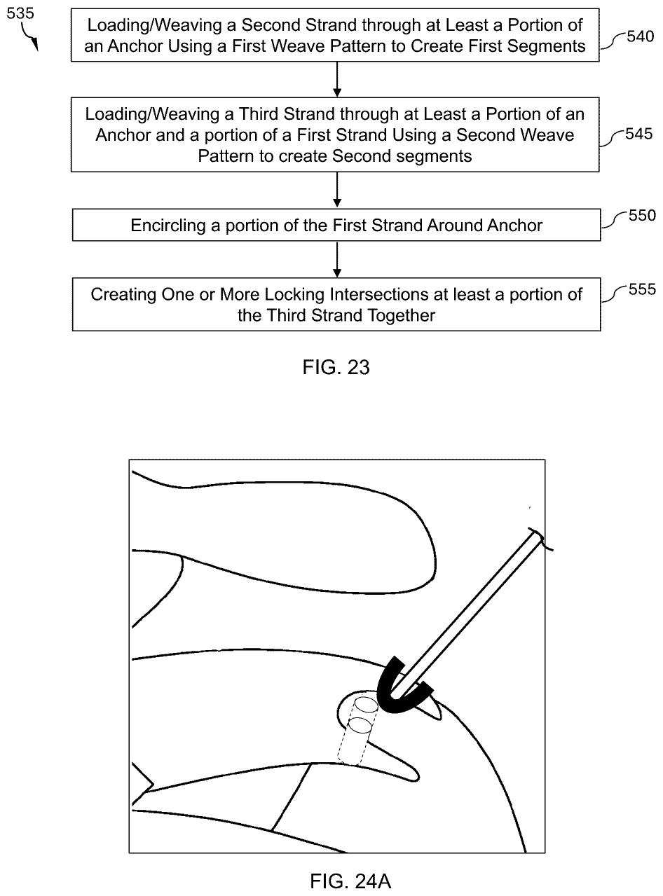

With reference to FIGS. 13A-13C and 23, the method 445 of creating a sixth suture anchor system 225 comprises the steps of: weaving a portion of a second strand through at least a portion of an anchor in a first weave pattern to create one or more first external segments 540, the first one or more first external segments extend from or contact the anchor outer surface; weaving a portion of a third strand through at least a portion of an anchor in a second weave pattern to create one or more second external segments 545, the one or more second external segments extend from or contact a portion of the first strand surface; encircling a portion of the first strand around the outer surface of the anchor 550; and intersecting the first free end and the second free end of the third strand to create one or more intersecting points 555, the intersecting points comprises locking intersecting points. The encircling step may comprise at least half-rotation around the outer surface of the anchor or greater. The encircling step may further comprise extending a portion of the first strand below or under the one or more first external segments.

### Method of Attaching Tissue to Bone

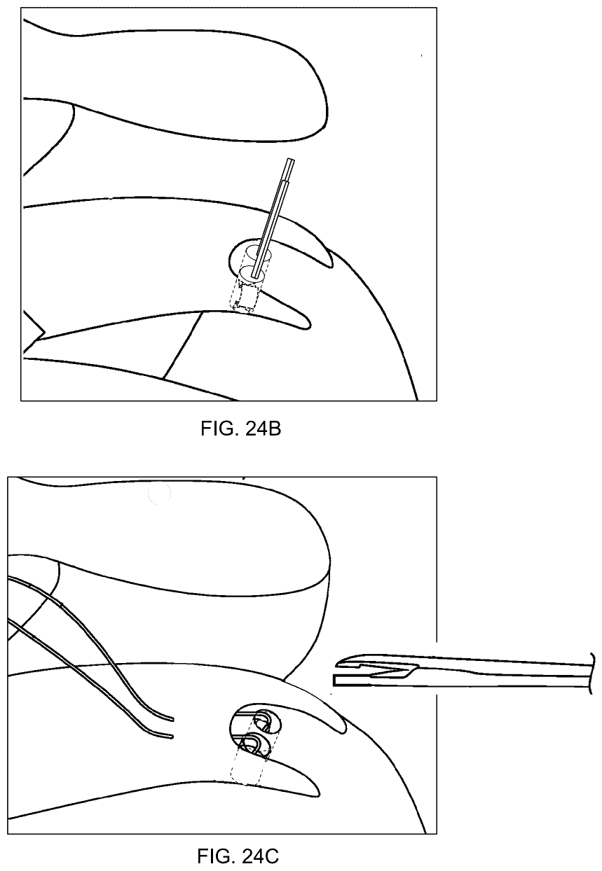

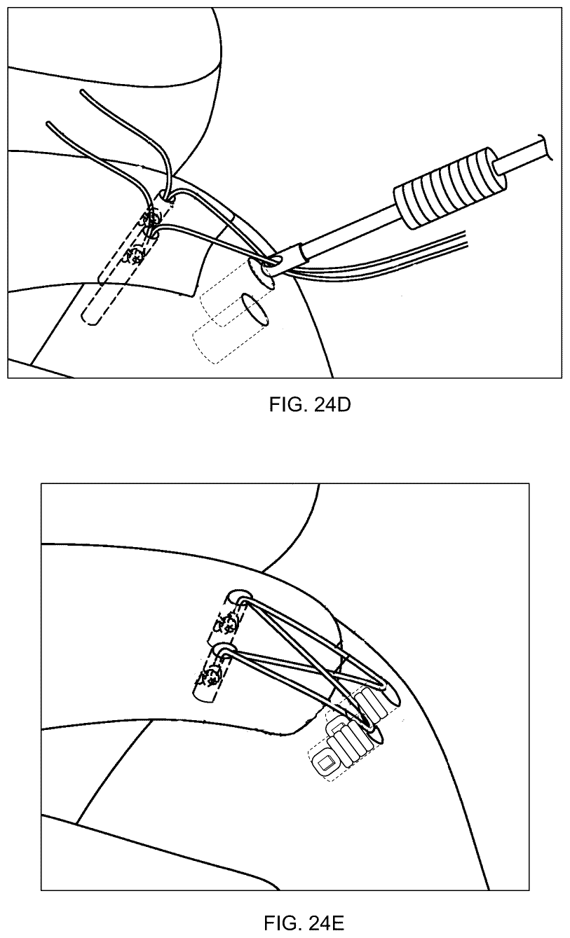

FIGS. 24A-24E schematically illustrate an exemplary method for attaching tissue to bone using one or more soft anchor assemblies. Either of the soft anchor assemblies can be utilized to attach tissue to bone. The soft anchor assemblies can be utilized in single-row techniques, double-row techniques, or any bridging technique to attach tissue to bone. In this non-limiting embodiment, the method is used to repair a torn rotator cuff. However, other repairs are also contemplated.

In one embodiment, the method of attaching tissue to bone using a medial row of suture anchor assemblies comprising the steps of: preparing a portion of a bone to create a first row of bone sockets; deploying a first one or more fixation devices into each first row of bone sockets; passing at least a first or second ends of the first and/or second strands of the first fixation device through a portion of the tissue; preparing a portion of the bone to create a second row of bone sockets; and deploying a second one or more fixation devices and the remaining first or second ends of the first or second strands of the first fixation device into an orientation within each of the second row of bone sockets.

The step of preparing a portion of a bone to create a first row of bone sockets may include the step of creating a bleeding bed and/or drilling into the bone to create a first row of bone sockets. A bleeding bed may be created adjacent to the tissue to enhance healing of the tissue to bone. In one nonlimiting embodiment, the tissue is a rotor cuff and the bone is a humerus. The bone is further prepared for receiving a medial row of fixation devices by drilling bone sockets. FIG. 24A depicts a portion of the bone comprising a first row of bone sockets. The sockets may be created by using a tool, such as a punch, adjacent to the articular margin of the bone. In one embodiment, the bone sockets are formed at a 45° “deadman” angle. The first row of bone sockets may include a medial row, the first row of bone sockets is sized and configured to receive one or more of the first fixation devices. The first one or more fixation devices may include one or more soft suture assemblies.

The step of deploying a first one or more fixation devices into each first row of bone sockets may comprise the step of: hammering the first one or more fixation devices; and/or tensioning the ends of the suture strands to set the anchor. The first one or more fixation devices comprises a first one or more soft anchor assemblies, the first one or more soft anchor assemblies are then inserted into the bone sockets (see FIG. 24A). The ends of the suture strands of each of the suture anchor assemblies are tensioned to compress or bunch together the anchor or anchor body to form an anchoring cluster within the bone sockets (see FIG. 24B). A first row of bone sockets may comprise a first bone socket and a second bone socket. The first row of bone sockets may comprise a first bone socket, a second bone socket and a third bone socket. Alternatively, a first row of bone sockets may comprise a plurality of bone sockets. This step may be repeated for each bone socket within the row.

The step of passing at least a first or second ends of the first and/or second strands of the first fixation device through a portion of the tissue may comprise the steps of loading the first and/or second ends of the first and/or second strands into a suture passer tool and/or tensioning the first and/or second strands. As shown in FIG. 24C, free ends of the strands of each suture anchor assembly of the medial row may next be passed through the tissue. Tensioning may be applied to the first and/or second strands so that the tissue is reduced and compressed against the bone, and once a desired tissue tension is achieved, the knotless anchors are ready to be inserted into the second row of bone sockets to complete the repair. Because the flexible strands are locked relative to the anchor of each suture anchor assembly, each free end of each first or second strand may be tensioned individually and prior to insertion of the second fixation device of the lateral row.

The step of preparing a portion of the bone to create a second row of bone sockets may include the step of creating a bleeding bed and/or drilling into bone to create a second row of bone sockets. The second row of bone sockets comprises a lateral row of bone sockets, the lateral row of bone sockets are sized and configured to receive a portion of the second one or more fixation devices. The second row of bone sockets may be prepared laterally from the medial row of anchors and slightly distal to the greater tuberosity of the bone. One free end from the flexible strand of each soft anchor assembly of the medial row may be connected to each knotless anchor of the lateral row. For example, in one embodiment, the free ends are loaded through a portion, such as an eyelet, of each knotless anchor. The second one or more fixation devices may include one or more knotless anchors. In one embodiment, the knotless anchors include SwivelLock® anchors. In another embodiment, the knotless anchors include PushLock® anchors. However, other knotless anchors may also be utilized.

The step of deploying a second one or more fixation devices and the first or second ends of the first or second strands of the first fixation device into an orientation within one of the second row of bone sockets may comprise the steps of retrieving a first or second free end from the first or second strand; creating the second row of bone sockets. The deployment or insertion step configures the first and/or second strands in a crisscross pattern (see FIG. 24E) or different orientation that provides a desired surface area for optimized compression. Any crisscross pattern using any number of fixations devices and strands can be achieved by using the soft anchor assemblies in a bridging technique. The orientation angles may include 0 degrees to 75 degrees.

### Testing Results

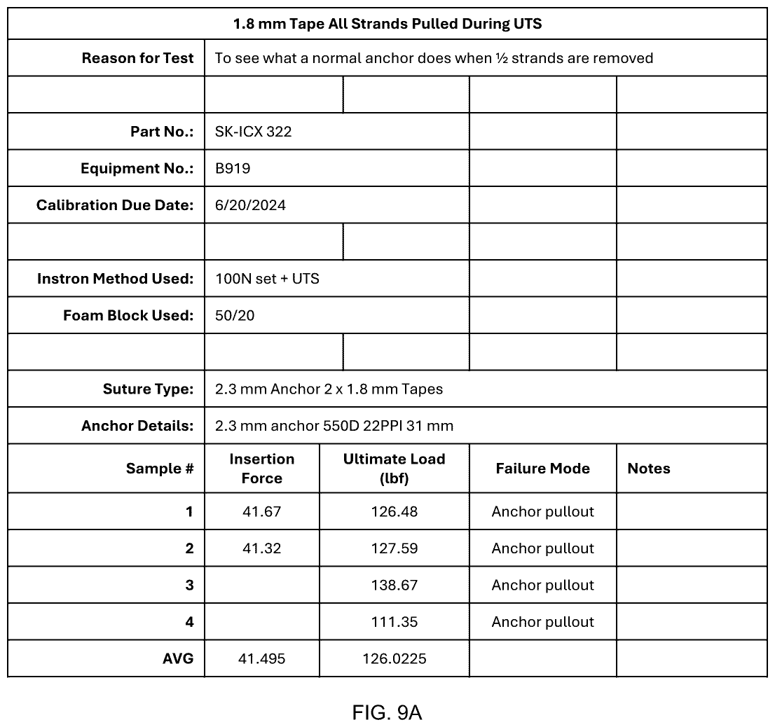

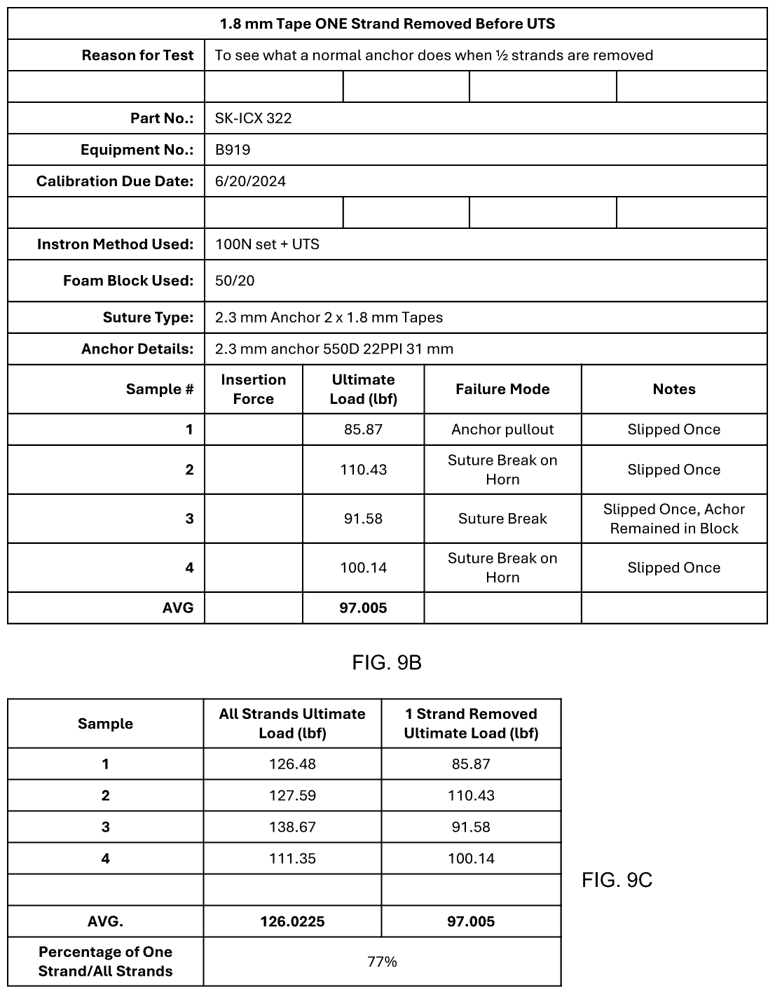

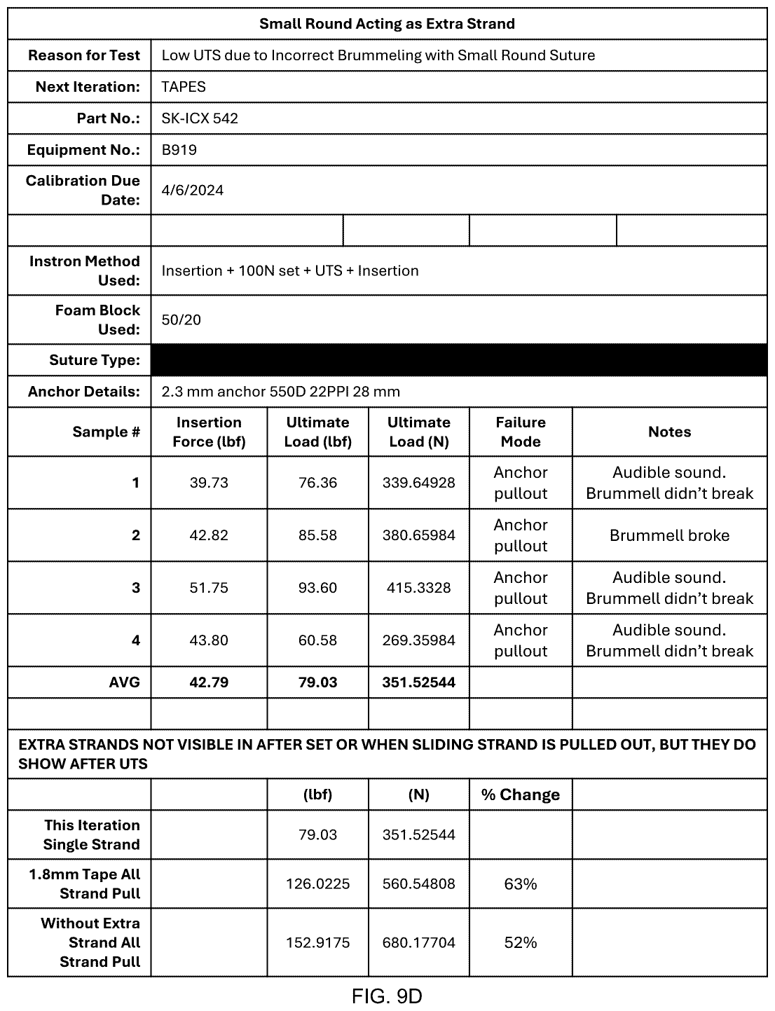

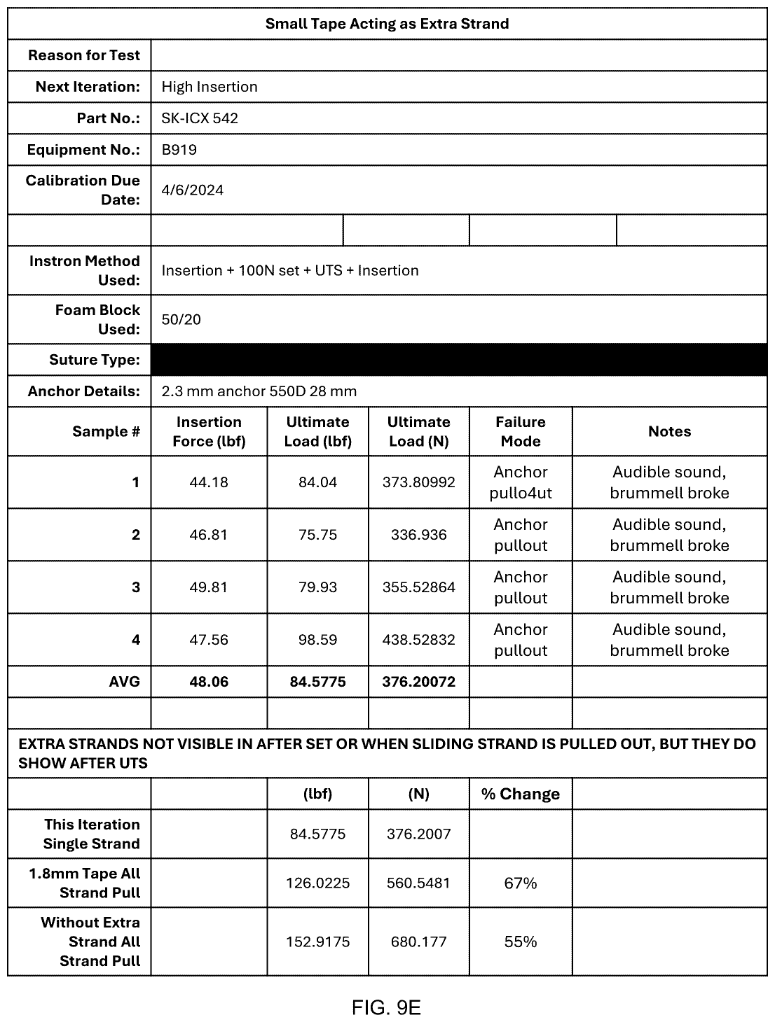

With reference to FIGS. 9A-9E, the tables illustrate insertion force and ultimate tensile pull strength of a control suture assembly and of the fifth embodiment suture anchor system 150 of FIGS. 6A-6B, 7A-7D, and 8A-8M. During surgery, it may be an option for the surgeon to remove the sliding strand or the second strand after insertion or deployment of the suture anchor assembly into the bone openings. The removal of the sliding strand may reduce the overall diameter or expansion of the suture assembly within the bone opening openings, which may result in a decreased pull-out strength. The testing compares the control suture anchor assembly insertion force and ultimate pull-out strength in a simulated in-vitro environment before and after the sliding strand is removed against another embodiment suture anchor assembly.

Table 1 below summarizes the data. The control from FIG. 9A shows that the control with all strands intact exhibited an average ultimate load or pull-out strength of 126.02 lbf, and 97.00 lbf when one of the strands of the suture assembly was removed. This calculates to 77% change, or the suture assembly maintaining 77% strength after one of the strands were removed. The suture assembly with a small round suture, the all strands intact exhibited an average ultimate pull-out strength of 152.92 lbf, and 79.03 lbf with one of the strands of the suture assembly was removed. This resulted in in a 52% change from the “all strands intact” and a 63% change from all strands intact of the control. The suture assembly with a flat suture, the all strands intact exhibited an average ultimate pull-out strength of 152.92 lbf, and 84.58 lbf with one strand removed from the suture assembly. This resulted in a 55% change from all strands intact and a 67% change from all strands intact of the control.

TABLE 1
Insertion and Pull-Out Strength

| | | Avg. Ultimate | Avg. | | |
|---|---|---|---|---|---|
| | Avg. Ultimate | Load (lbf) | Insertion | % Change | % Change |
| | Load (lbf) | (One Strand | Force | from All | from |
| Sample | (All Strands) | Removed) | (lbf) | Strands | Control |
| Control (FIG. 13A) | 126.02 | 97.00 | 41.50 | 77% | N/A |
| Suture Anchor Assy - | 152.92 | 79.03 | 42.79 | 52% | 63% |
| Small Round (FIG. 13B) | | | | | |
| Suture Anchor Assy - | 152.92 | 84.58 | 48.06 | 55% | 67% |
| Small Tape (FIG. 13C) | | | | | |

The data also shows that the suture assemblies containing a small round suture compared to a small tape suture (e.g., flat suture) showed minimal pull-out strength % change. The data infers that using a small round suture or small flat suture may not significantly affect the pull-out strength.

### Example Embodiments

In one embodiment, the suture anchor system comprises (1^{st} embodiment): a first strand, the first strand comprises a first strand longitudinal axis, a first body, a first free end and a second free end, the first body extending between the first and second free ends, the first body comprising and a first body opening, the first body opening extending transverse to the first strand longitudinal axis, the first body opening being positioned between the first and second free ends; and an anchor, the anchor comprising a tubular anchor body, a first end, a second end and an anchor material, the tubular anchor body including a lumen extending between the first and second ends, at least a portion of the anchor extending through the first body opening of the first strand; at least a portion of the first free end of the first strand extending through the tubular anchor body adjacent to the first end of the tubular anchor body, at least a portion of the second free end of the first strand extending through a portion of the tubular anchor body adjacent to the second end of the tubular anchor body.

In another embodiment, the suture anchor system comprises a first strand, the first strand comprises a first strand longitudinal axis, a first body, a first free end and a second free end, the first body extending between the first and second free ends, the first body comprising a first body opening, the first body opening extending transverse to the first strand longitudinal axis, the first body opening being positioned between the first and second free ends; and an anchor, the anchor comprising a tubular anchor body, a first end, a second end, the tubular anchor body including a lumen extending between the first and second ends, a first anchor opening and a second anchor opening, at least a portion of the anchor extending through the first body opening of the first strand at a set location; the first free end of the first strand extending through a first anchor opening and a second free end of the first strand extending through a second opening, the first strand being movable from an unlocked position to a locked position, the locked position locks the first strand relative to the anchor.

In another embodiment, the suture anchor system comprises: a first strand, the first strand comprises a first strand longitudinal axis, a first body, a first free end and a second free end, the first body extending between the first and second free ends, the first body comprising a first body opening, the first body opening extending transverse to the first strand longitudinal axis, the first body opening being positioned at a set location between the first and second free ends; and an anchor, the anchor comprising a tubular anchor body, a first end, a second end, the tubular anchor body including a lumen extending between the first and second ends, a first anchor opening, a second anchor opening, a third anchor opening and a fourth anchor opening, at least a portion of the anchor extending through the first body opening of the first strand at a set location; the first free end of the first strand extending through a first anchor opening at a first distance from the set location and a second anchor opening in a first portion of the anchor, the second free end of the first strand extending through a third anchor opening at a second distance from the set location and a fourth anchor opening in a second portion of the anchor.

In another embodiment, the suture anchor system comprises a first strand, the first strand comprises a first strand longitudinal axis, a first free end, a second free end, and at least one opening, the at least one opening extending transverse to the first strand longitudinal axis, the at least one opening being positioned at a set location between the first and second free ends; and an anchor, the anchor comprising a tubular anchor body, a first end, a second end and an outer diameter, the tubular anchor body including a lumen extending between the first and second ends; a first or second end of the first strand extending through the at least one opening of the first strand, the at least one opening being positioned at or adjacent to the outer diameter of the anchor, the first end of the first strand extending through the first anchor opening and the second end of the first strand extending through the second anchor opening.

In another embodiment, the suture anchor system comprises a first strand, the first strand comprises a first strand longitudinal axis, a first free end, a second free end, and at least one opening, the at least one opening extending transverse to the first strand longitudinal axis, the at least one opening being positioned at a set location between the first and second free ends; and an anchor, the anchor comprising a tubular anchor body, a first end, a second end and an outer diameter, the tubular anchor body including a lumen extending between the first and second ends, a first anchor opening, and a second anchor opening; a first or second end of the first strand extending through the at least one opening of the first strand, the at least one opening being positioned at or adjacent to the outer diameter of the anchor, the first end of the first strand extending through the first anchor opening and the second end of the first strand extending through the second anchor opening.

In another embodiment, the suture anchor system comprises: a first strand, the first strand comprises a first strand longitudinal axis, a first free end, a second free end, and at least one opening, the at least one opening extending transverse to the first strand longitudinal axis, the at least one opening being positioned at a set location between the first and second free ends; and an anchor, the anchor comprising a tubular anchor body, a first end, a second end and an outer diameter, the tubular anchor body including a lumen extending between the first and second ends, a first anchor opening, and a second anchor opening; a first or second end of the first strand extending through the at least one opening of the first strand, the at least one opening being positioned at or adjacent to the outer diameter of the anchor to create at least one intersection point, the first end of the first strand extending through the first anchor opening in a first portion of the tubular anchor body and the second end of the first strand extending through the second anchor opening in a second portion of the tubular anchor body.

In another embodiment, the suture anchor system comprises: a first strand, the first strand comprises a first strand longitudinal axis, a first free end, a second free end, and one or more openings, the one or more openings extending transverse to the first strand longitudinal axis, the one or more openings being positioned at one or more set locations between the first and second free ends; and an anchor, the anchor comprising a tubular anchor body, a first end, a second end and an outer diameter, the tubular anchor body including a lumen extending between the first and second ends, a first anchor opening, a second anchor opening; a portion of the first strand being received in a portion of the lumen, a first or second end of the first strand extending through each of the one or more openings of the first strand to create one or more locking intersection points, the one or more locking intersection points being positioned at or adjacent to the outer diameter of the anchor, the first end of the first strand extending through a first anchor opening adjacent to the first end of the anchor and the second end of the first strand extending through a second anchor opening adjacent to the second end of the anchor.

In another embodiment, the suture anchor system comprises: a first strand, the first strand comprises a first strand longitudinal axis, a first strand material, a first free end, a second free end, and at least one strand opening, the at least one opening extending transverse to the first strand longitudinal axis, the at least one opening being positioned at a set location between the first and second free ends; an anchor, the anchor comprising a tubular anchor body, a first end, a second end, an outer diameter and an anchor material, the tubular anchor body including a lumen extending between the first and second ends, one or more anchor openings; and a second strand, the second strand being slidable relative to the anchor, the second strand comprising a second strand material, at least a portion of the second strand extending through the one or more anchor openings to create one or more loop segments, the one or more loop segments being positioned at or adjacent to the outer diameter of the tubular body of the anchor; at least a portion of the first stand extending through the one or more loop segments.

In another embodiment, the suture anchor system comprises a first strand, the first strand comprises a first strand longitudinal axis, a first strand material, a first free end, a second free end, and at least one strand opening, the at least one opening extending transverse to the first strand longitudinal axis, the at least one opening being positioned at a set location between the first and second free ends; an anchor, the anchor comprising a tubular anchor body, a first end, a second end, an outer diameter and an anchor material, the tubular anchor body including a lumen extending between the first and second ends, one or more anchor openings; and a second strand, the second strand being slidable relative to the anchor, the second strand comprising a second strand material, at least a portion of the second strand extending through the one or more anchor openings to create one or more loop segments, the one or more loop segments being positioned at or adjacent to the outer diameter of the tubular body of the anchor; at least a portion of the first stand extending through the one or more loop segments to create one or more locking intersection points, the second strand being movable from an unlocked position to a locked position, the locked position making the first strand fixed relative to the anchor at or adjacent to the locking intersection points.

In another embodiment, the suture anchor system comprises a first strand, the first strand comprises a first body, a first free end and a second free end, the first body extending between the first and second free ends, the first body comprising a first body opening and a second body opening, the first body opening and second body opening extending transverse to a first strand longitudinal axis, the first body opening and second body opening being positioned between the first and second free ends, the first body opening and the second body opening being spaced apart at a set distance; an anchor, the anchor comprising a tubular anchor body, a first end, a second end and a material, the tubular anchor body including a lumen extending between the first and second ends and an outer surface, the anchor extending through the first and second body opening of the first strand to create a first tail and a second tail; a second strand, the second strand being slidable relative to the anchor; the second strand extending through at least a portion of the anchor in a first pattern; a third strand, the third strand including a first free end and a second free end, at least a portion of the third strand extending through at least a portion of the anchor in a second pattern, the first free end of the third strand extending through a portion of the first tail of the first strand and the second first free end of the third strand extending through a portion of the second tail of the first strand, the first free end of the third strand and the second free end of the third strand intersecting to create a locking splice.

For any preceding example embodiments, wherein the suture anchor assembly further comprises a second strand or sliding strand, the second strand being slidable relative to the anchor.

For any preceding example embodiments, wherein the first strand and the anchor comprise a material, the material includes a soft, flexible suture material.

For any preceding example embodiments, wherein the first strand comprises a flat suture configuration or a flat-to-round suture configuration.

For any preceding example embodiments, wherein the first strand comprises suture tape and the second strand comprises a suture.

For any preceding example embodiments, wherein the first pattern includes the second strand extending over at least a portion of the outer surface of the anchor and through a portion of the lumen at one or more set locations to create one or more loop segments.

For any preceding example embodiments, the second pattern comprises the same pattern as the first pattern. Alternatively, the second pattern comprises a different pattern than the first pattern.

For any preceding embodiments, the locking splice comprises a double, triple or quadruple Brummel splice. Alternatively, the locking splice may comprise a back splice, a chain splice, an eye splice and/or any combination thereof.

In another embodiment, a method of creating a suture anchor system comprises the steps of: Piercing a first strand using an anchor at a set location; Measuring a first distance from the set location adjacent to first end of the anchor to load a first end of the first strand; Extending the first end of the first strand through a portion of the anchor the at the first distance; Measuring a second distance from the set location adjacent to second end of the anchor to load a second end of the first strand; Extending the second end of the first strand through a portion of the anchor at the second distance; Tensioning the first and second ends of the first strand to lock the first strand relative to the anchor.

In another embodiment, the method of creating a suture anchor system comprises the steps of: extending an anchor through a first strand opening of a first strand to create a locking intersection region, the first strand opening being transverse to a longitudinal axis of the first strand; loading a first end of the first strand through a first anchor opening at a first set distance from the locking intersection region, and extending a first end of the first strand through a second anchor opening; loading a second end of the first strand through a second anchor opening at a second set distance from the locking intersection region, and extending a first end of the first strand through a second anchor opening; tensioning the first and second ends of the first strand to fix, cinch or lock the first strand relative to the anchor.

In another embodiment, the method of creating a suture anchor system comprises the steps of: loading a first end of a first strand through a first opening on an anchor; loading a second end of a first strand through a second opening an the anchor; extending a first or second end of the first strand through at least one first strand opening to create at least one locking intersection, the at least one locking intersection disposed at or adjacent to an outer diameter of the anchor; extending the first end through a third opening disposed at a first end of the anchor and a second end through a fourth opening disposed at a second end of the anchor; tensioning the first and second ends of the first strand to fix, cinch or lock the first strand relative to the anchor at the at least one locking intersection.

In another embodiment, the method of creating a suture anchor system comprises the steps of: passing a portion of the first strand through a portion of a lumen of an anchor; piercing a first end or second end of the first strand through one or more first strand openings to create one or more locking intersection regions, the one or more locking intersection regions disposed at or adjacent to an outer diameter of the anchor; extending a first end of the first strand through a first anchor opening positioned at the first end of the anchor; Extending a second end of the first strand through a second anchor opening positioned at the second end of the anchor; Tensioning the first and second end of the first strand to fix, cinch or lock the first strand relative to the anchor at the least one locking intersection regions.

In another embodiment, the method of creating a suture anchor system comprises the steps of: piercing a portion of a first strand with an anchor at a set location; passing a second strand through one or more anchor body openings to create one or more loop segments, the one or more loop segments positioned at or adjacent to an outer diameter of the anchor, the second strand being slidable relative to the anchor; weaving the first strand through the one or more loop segments to create one or more locking intersection locations; tensioning the second strand to lock, cinch or wedge the first strand relative to the anchor at the one or more intersection locations.

In another embodiment, the method of creating a suture anchor system comprises the steps of: extending an anchor through a first strand opening of the first strand, the first strand opening positioned between the first and second ends of the first strand; passing a second strand through a plurality of anchor body openings of the anchor to create at least one loop segment, the at least one loop segment positioned at or adjacent to an outer diameter of the anchor, the second strand being slidable relative to the anchor; weaving the first strand through the at least loop segment to create at least one locking intersection locations; tensioning the second strand to lock, cinch or wedge the first strand relative to the anchor at the least one intersection locations.

In another embodiment, the method of creating a suture anchor system comprises the steps of: Extending an anchor through a first strand opening and a second strand opening of the first strand to create a first tail and a second tail, the first strand opening and second strand opening positioned between a first and second free ends of the first strand; loading or weaving a second strand through an anchor in a first pattern, the second strand being slidable relative to the anchor; loading or weaving a third strand through the anchor in a second pattern to create a first free end and a second free end; and piercing the first free end of the third strand through a portion of the first tail of the first strand and the second free end of the third strand through a portion of the second tail of the first strand; and intersecting the first free end of the third strand and the second free end of the third strand at one or more locations to create one or more locking splices.

In another embodiment, the method of creating a suture anchor system comprises the steps of: providing an suture construct, the suture construct includes an anchor, a fixation strand and a fixed strand, the anchor comprises a lumen, a first opening, and a second opening, the anchor further includes an outer surface and a sidewall, the lumen disposed between the first and second opening; weaving at least a portion of the fixation through the sidewall of the anchor in a first weave pattern to create one or more fixation strand segments, the fixation strand segments extends from the outer surface of the anchor; piercing the fixed strand in at least two locations to create a bridge segment, the bridge segment extends from an outer surface of the fixed strand; creating a first splice region by extending a first portion of the fixed strand through a first portion of the fixation strand and a second splice region by extending a second portion of the fixed strand through a second portion of the fixation strand, the bridge segment disposed between the first splice region and the second splice region; wrapping or encircling the outer surface of the anchor with the first splice region and the second splice region at least a half-rotation; securing a first end and a second end of the fixation strand together. The step of providing a suture anchor assembly or construct further comprises a sliding strand. The first method of assembling a first embodiment of a suture anchor assembly or construct further comprises the step of: weaving at least a portion of the sliding strand through the sidewall of the anchor in a second weave pattern to create one or more sliding strand segments, the sliding strand segments extends from the outer surface of the anchor. The second weave pattern is the same or different than the first weave pattern.

In another embodiment, the method of creating a suture anchor system comprises the steps of: providing an anchor, a fixation strand and a fixed strand, the anchor comprises a lumen, a first opening, and a second opening, the anchor further includes an outer surface and a sidewall, the lumen disposed between the first and second opening; weaving at least a portion of the fixation strand through the sidewall of the anchor in a weave pattern to create one or more fixation strand segments, the one or more fixation strand segments extends from the outer surface of the anchor; extending a first end of the fixation strand through or below at least one fixation strand segment after the first end of the fixation strand encircles the outer surface of the anchor in at least a ¼ rotation; piercing the fixed strand in at least two locations to create a bridge segment, the bridge segment extends from an outer surface of the fixed strand; wrapping or encircling the outer surface of the anchor with the first splice region and the second splice region at least a half-rotation; and securing a first end and a second end of the fixation strand together. The step of providing a suture anchor assembly or construct further comprises a sliding strand. The second method of assembling a second embodiment of a suture anchor assembly or construct further comprises the step of: weaving at least a portion of the sliding strand through the sidewall of the anchor in a second weave pattern to create one or more sliding strand segments, the sliding strand segments extends from the outer surface of the anchor. The second weave pattern is the same or different than the first weave pattern.

In another embodiment, the method of creating a suture anchor system comprises the steps of: providing an anchor, a fixation strand and a fixed strand, the anchor comprises a lumen, a first opening, and a second opening, the anchor further includes an outer surface and a sidewall, the lumen disposed between the first and second opening; weaving at least a portion of the fixation strand through the sidewall of the anchor in a weave pattern to create one or more fixation strand segments, the one or more fixation strand segments extends from the outer surface of the anchor; piercing the fixed strand transversely at a first and a second location; wrapping or encircling the outer surface of the anchor with a portion of the fixation strand at least one rotation; wrapping or encircling the outer surface of the with a portion of the fixed strand at least a one-half rotation; securing a first and second end of the fixation strands together.

For any preceding example method embodiments, wherein the method further comprises the step of tucking the first free end of the third strand into a first end of the anchor and the second free end of the third strand into a second end of the anchor.

For any preceding example method embodiments, wherein the method may further comprise the step of trimming the first free end and/or the second free end of the third strand.

For any preceding example method embodiments, wherein the one or more locking splices may comprise a double, triple or quadruple Brummel splice.

For any preceding example method embodiments, wherein the first strand is a flat suture and/or round-to-flat suture.

For any preceding example method embodiments, wherein the flat suture comprises suture tape.

For any preceding example method embodiments, wherein the second strand comprises flat or round suture.

For any preceding example method embodiments, wherein the third strand comprises flat or round suture.

For any preceding example method embodiments, wherein the third strand comprises a third strand length, the third strand length is smaller than a first strand length or a second strand length.

For any preceding example method embodiments, wherein the first free end of the third strand comprises a first free end length and the second free end of the third strand comprises a second free end length, the first free end length and the second free end length comprises a length of at least 5 mm.

For any preceding example method embodiments, wherein the third strand length comprises a length of at least 50× a third strand diameter.

I/We claim:

1. The suture anchor system comprises:
an anchor, the anchor comprises a lumen, a first end, and a second end, the anchor further includes an outer surface and a sidewall, the lumen disposed between the first and second ends;
a fixation strand, the fixation strand comprising a first free end and a second free end, at least a portion of the fixation strand passes through sidewall of the anchor in a first location to exit out a second location to create at least one fixation strand segment, the at least one fixation strand segment extend from the outer surface of the anchor;
a first strand, the first strand being pierced transversely by the fixation strand at a first location and a second location to create at least one strand segment, the fixation strand encircling the outer surface of the anchor by at least 1 rotation and extending below one or more fixation strand segments, the first end and the second end of the fixation strand being coupled together.

2. The method of making the suture anchor system comprises the steps of:
weaving a portion of a second strand through at least a portion of an anchor in a first weave pattern to create one or more first external segments, the first one or more first external segments extend from or contact the anchor outer surface;
weaving a portion of a third strand through at least a portion of an anchor and ta first strand in a second weave pattern to create one or more second external segments, the one or more second external segments extend from or contact a portion of the first strand surface;
encircling a portion of the first strand around the outer surface of the anchor; and
intersecting the first free end and the second free end of the third strand to create one or more intersecting points, the intersecting points comprises locking intersecting points.
